(19) United States
(12) Patent Application Publication
(10) Pub. No.: US 2024/0132978 A1
(43) Pub. Date: Apr. 25, 2024

(54) SEQUENCING MICROBIAL CELL-FREE NUCLEIC ACIDS TO DETECT INFLAMMATION, SECONDARY INFECTION, AND DISEASE SEVERITY

(71) Applicants: Karius, Inc., Redwood City, CA (US); University of Pittsburgh - Of the Commonwealth System of Higher Education, Pittsburgh, PA (US)

(72) Inventors: Asim Ahmed, Redwood City, CA (US); Radha Duttagupta, Redwood City, CA (US); Georgios D. Kitsios, Pittsburgh, PA (US)

(21) Appl. No.: 18/338,128

(22) Filed: Jun. 19, 2023

Related U.S. Application Data
(63) Continuation of application No. PCT/US2021/064445, filed on Dec. 20, 2021.
(60) Provisional application No. 63/128,552, filed on Dec. 21, 2020.
(60) Provisional application No. 63/199,497, filed on Jan. 3, 2021.
(60) Provisional application No. 63/139,245, filed on Jan. 19, 2021.

(51) Int. Cl. C12Q 1/70; C12Q 1/689; C12Q 1/6895; G01N 33/68

(52) U.S. Cl. CPC C12Q 1/701; C12Q 1/689; C12Q 1/6895; G01N 33/6863; G01N 33/6893; C12Q 1/6874

(57) ABSTRACT

Described herein is a method of detecting secondary infection in a patient, particularly a patient with a primary infection that is a pneumonia, a COVID-19 infection, or a COVID-19 pneumonia. In some cases, the secondary infection is a secondary bacterial infection, e.g., secondary bacterial pneumonia. In some cases, the methods provided herein detect a hyper-inflammatory response or severity of disease, e.g., indicating a severe COVID-19 infection, or provide a risk of death from a disease (e.g., COVID-19). This disclosure also provides method of detecting a localized respiratory infection in a subject by quantifying microbial cell-free nucleic acids (e.g., mdfDNA) from plasma from the subject. In some cases, the subject is not bacteremic when plasma is collected from the subject. This disclosure also provides systems, such as nucleic acid-sequencing systems with increased reliability for detecting secondary infections, particularly in patients with culture-negative pneumonia.

### CROSS-REFERENCE

This application is a continuation of International Application No. PCT/US2021/064445, filed Dec. 20, 2021, which claims the benefit of U.S. Provisional Patent Application No. 63/128,552, filed Dec. 21, 2020, U.S. Provisional Patent Application No. 63/199,497, filed Jan. 3, 2021, and U.S. Provisional Patent Application No. 63/139,245, filed Jan. 19, 2021, which are herein incorporated by reference in their entireties.

### BACKGROUND

Severe COVID-19 pneumonia can be complicated by secondary bacterial or fungal infections, but their clinical distinction from isolated SARS-CoV-2 infection is challenging, especially with the more restricted practices regarding invasive diagnostics in patients with COVID-19. We sought to comprehensively screen for secondary infections by DNA pathogens (bacterial, fungal or viral) with a non-invasive, culture-independent metagenomic approach (microbial cell-free DNA sequencing—mcfDNA-Seq), and also examine for the biologic impact of circulating mcfDNA on the host response in COVID-19.

Variability in host inflammatory response has emerged as a key predictor of outcome in critically ill patients. Elevated biomarkers of host innate immunity and inflammation upon admission to the Intensive Care Unit (ICU) have been consistently associated with worse outcomes in patients with severe pneumonia and acute respiratory distress syndrome (ARDS). Little is known about the specific stimuli and triggers of this inflammatory response, but recent research implicates variation in the lung microbiome in patients with acute respiratory failure. Low community diversity and high abundance of pathogenic bacteria in the respiratory tract possibly correlate with elevated inflammatory biomarkers and worse clinical outcomes. It is unclear whether this early systemic inflammatory response reflects local interactions between microbes and immune cells in the alveolar space or systemic activation of innate immunity from circulating pathogen-associated molecular patterns (PAMPs) that leak from the injured alveolar epithelium. Such distinction is important for understanding severe pneumonia pathogenesis and clarifying causal mechanisms for circulating PAMPs.

The advent of ultra-sensitive, plasma metagenomic sequencing for circulating microbial cell-free DNA (mcfDNA) offers the opportunity to study the impact of a PAMP (mcfDNA) on systemic host-responses in pneumonia.

### SUMMARY

In one aspect, a method of detecting a secondary infection in a subject with a first infection is provided, comprising: (a) preparing a plasma sample from blood obtained from the subject with the first infection, wherein the plasma sample comprises microbial cell-free nucleic acids (mcfNA) from at least two different microbes; (b) producing a sequencing library comprising mcfNA attached to adapters; (c) measuring an amount of total mcfNA in the plasma sample by performing next generation sequencing on the sequencing library comprising the mcfNA attached to adapters, wherein the total mcfNA comprises mcfNA from at least two different microbes; (d) comparing the amount of total mcfNA comprising mcfNA from at least two different microbes to a threshold amount of total mcfNA; and (e) detecting a secondary infection that is different from the first infection when the amount of total mcfNA comprising mcfNA from at least two different microbes exceeds the threshold amount of total mcfNA.

In another aspect, a method of detecting a secondary infection in a subject with a first infection is provided, comprising: (a) preparing a plasma sample from blood obtained from the subject with the first infection, wherein the plasma sample comprises microbial cell-free nucleic acids (mcfNA) from at least two different microbes; (b) measuring an amount of total mcfNA in the plasma sample by performing next generation sequencing, wherein the total mcfNA comprises mcfNA from at least two different microbes; (c) comparing the amount of total mcfNA comprising mcfNA from at least two different microbes to a threshold amount of total mcfNA; and (d) detecting a secondary infection that is different from the first infection when the amount of total mcfNA comprising mcfNA from at least two different microbes exceeds the threshold amount of total mcfNA.

In yet another aspect, a method of treating a secondary infection in a subject with a first infection is provided, the method comprising: (a) collecting a blood sample from the subject with the first infection; (b) detecting a secondary infection when an amount of total microbial cell-free nucleic acids (mcfNA) comprising mcfNA from at least two microbes in the blood sample exceeds a threshold amount of total mcfNA, wherein the amount of total mcfNA is calculated by next generation sequencing; and (c) administering a therapeutic drug to the subject with the first infection in order to treat the secondary infection. In some cases, the method further comprises (d) repeating (a), (b), and (c) until the amount of total mcfNA in the blood decreases to a value at or below the threshold amount of total mcfNA.

In yet another aspect, a method of treating a secondary infection in a subject with a first infection is provided, the method comprising: (a) collecting a blood sample from the subject with the first infection; and (b) detecting a secondary infection when an amount of total microbial cell-free nucleic acids (mcfNA) comprising mcfNA from at least two microbes in the blood sample exceeds a threshold amount of total mcfNA, wherein the amount of total mcfNA is calculated by next generation sequencing.

In any of the preceding methods, in some embodiments, the first infection is a COVID-19 infection. In any of the preceding methods, in some embodiments, the first infection is a viral lung infection. In any of the preceding methods, in some embodiments, the first infection is COVID-19 pneumonia. In any of the preceding methods, in some embodiments, the secondary infection is a bacterial or fungal infection. In any of the preceding methods, in some embodiments, the method further comprises determining a presence of at least one bacterium, fungus, or parasite in the subject. In any of the preceding methods, in some embodiments, the first and secondary infections are respiratory infections caused by different microbes. In any of the preceding methods, in some embodiments, the first and second infections are pneumonia caused by different microbes. In any of the preceding methods, in some embodiments, the at least two microbes are respiratory pathogens. In any of the preceding methods, in some embodiments, the at least two microbes are at least two microbes from the group consisting of S. aureus, P. aeruginosa and K. pneumoniae. In any of the preceding methods, in some embodiments, the at least two microbes are at least two microbes listed in Table 2. In any of the preceding methods, in some embodiments, the at least two microbes are at least two respiratory pathogens listed in Table 2. In any of the preceding methods, in some embodiments, the first infection is culture-positive pneumonia. In any of the preceding methods, in some embodiments, the first infection is culture-negative pneumonia. In any of the preceding methods, in some embodiments, the at least two microbes comprise Candida. In any of the preceding methods, in some embodiments, the amount of total mcfNA is an aggregated amount of each type mcfNA in the sample. In any of the preceding methods, in some embodiments, the amount of total mcfNA is an aggregated amount of total bacterial mcfNA in the sample. In any of the preceding methods, in some embodiments, the amount of total mcfNA is an aggregated amount of total mcfNA from respiratory pathogens in the sample. In any of the preceding methods, the threshold amount of total mcfNA is an amount of mcfNA measured in plasma of a healthy or un-infected subject. In any of the preceding methods, in some embodiments, the amount of total mcfNA is measured by metagenomic next generation sequencing. In any of the preceding methods, in some embodiments, the mcfNA is mcfDNA. In any of the preceding methods, in some embodiments, the plasma or blood sample is spiked with a known concentration of synthetic normalization controls. In any of the preceding methods, in some embodiments, the mcfNA is extracted from the plasma of the subject. In any of the preceding methods, in some embodiments, a DNA sequencing library is constructed from the extracted mcfNA, and sequence reads are produced from the sequencing library. In any of the preceding methods, in some embodiments, the measuring the amount of mcfNA in the sample comprises (a) aligning the sequence reads with a microorganism database, wherein the microorganism library comprises more than 10,000 genomic reference sequences; (b) retaining reliable reads comprising alignments with high percent identity and high query coverage; (c) assigning relative abundances to each taxon based on the number of reliable reads and their alignments; (d) computing statistical significance values for each estimate of taxon abundance; (e) using taxon abundance to determine mcfNA concentration; and/or (f) using abundance of spiked synthetic normalization controls to calculate the molecules per microliter (MPM) value of mcfNA in the sample. In any of the preceding methods, in some embodiments, the microorganism library comprises at least 100, 200, 500, 750, 1000, 2000, 5000, 9000, 10000, or 15000 genomic reference sequences. In any of the preceding methods, in some embodiments, the method further comprises measuring levels of biomarkers of innate immunity or epithelial or endothelial injury in the plasma sample of the subject. In any of the preceding methods, in some embodiments, the biomarkers are selected from the group consisting of IL-6, IL-8, IL-10, RAGE, TNFR1, angiopoietin-2, procalcitonin, fractalkine, pentraxin-3, and ST2. In any of the preceding methods, in some embodiments, the biomarker is IL-8 or ST2. In any of the preceding methods, in some embodiments, the biomarker is procalcitonin or pentraxin-3. In any of the preceding methods, in some embodiments, the method further comprises comparing the amount of mcfNA in the patient with the biomarker levels using an algorithm to yield a test score. In any of the preceding methods, in some embodiments, the method further comprises administering a therapeutic drug to the patient based on the test score. In any of the preceding methods, in some embodiments, the therapeutic drug is optionally an antimicrobial drug, an antibiotic drug, or an antifungal drug. In any of the preceding methods, in some embodiments, the amount is measured in molecules per microliter of plasma (MPM). In any of the preceding methods, in some embodiments, the threshold amount of total mcfNA is greater than 400 MPM for all types of mcfNA in the sample. In any of the preceding methods, in some embodiments, the threshold amount of total mcfNA is greater than 600 MPM for total mcfNA in the sample when the total mcfNA is determined by aligning sequence reads to a genomic database comprising sequences from at least 100 different microbes. In any of the preceding methods, in some embodiments, the threshold amount of total mcfNA is greater than 4000 MPM for mcfNA from respiratory pathogens in the sample. In any of the preceding methods, the threshold amount of total mcfNA is greater than 4000 MPM when the total mcfNA is determined by aligning sequence reads to a genomic database comprising sequences from at least 100 different microbes. In any of the preceding methods, in some embodiments, the subject in (a) has received an empiric antibiotic. In any of the preceding methods, in some embodiments, the subject is not bacteremic. In any of the preceding methods, in some embodiments, the method further comprises adding synthetic nucleic acids to the plasma sample. In any of the preceding methods, in some embodiments, the method further comprises performing next generation sequencing of the synthetic nucleic acids. In any of the preceding methods, in some embodiments, the method further comprises attaching adapters to the cell-free nucleic acids in order to produce cell-free nucleic acids attached to the adapters. In any of the preceding methods, in some embodiments, the adapters are ligated to the cell-free nucleic acids. In any of the preceding methods, in some embodiments, the adapters are attached to the cell-free nucleic acids by a primer extension reaction. In any of the preceding methods, in some embodiments, the adapters comprise a sequence unique to the subject. In any of the preceding methods, in some embodiments, the method further comprises combining the cell-free nucleic acids attached to the adapters with cell-free nucleic acids obtained from a different subject. In any of the preceding methods, in some embodiments, the cell-free nucleic acids obtained from a different subject are attached to adapters that comprise a sequence unique to the different subject.

In yet another aspect, a method of detecting an inflammatory response in a patient is provided, comprising: (a) preparing a plasma sample from blood obtained from the patient, wherein the plasma sample comprises microbial cell-free nucleic acids (mcfNA); (b) producing a sequencing library comprising mcfNA attached to adapters; (c) measuring an amount of total mcfNA in the plasma sample, wherein the total mcfNA comprises mcfNA from at least two different microbes; (d) comparing the amount of the total mcfNA to a threshold amount of mcfNA; and (e) detecting an inflammatory response when the amount of total mcfNA exceeds the threshold amount of total mcfNA.

In yet another aspect, a method of detecting an inflammatory response in a patient is provided, comprising: (a) preparing a plasma sample from blood obtained from the patient, wherein the plasma sample comprises microbial cell-free nucleic acids (mcfNA); (b) measuring an amount of total mcfNA in the plasma sample, wherein the total mcfNA comprises mcfNA from at least two different microbes; (c) comparing the amount of the total mcfNA to a threshold amount of mcfNA; and (d) detecting an inflammatory response when the amount of total mcfNA exceeds the threshold amount of total mcfNA.

In yet another aspect, a method of treating an inflammatory response in a patient is provided, comprising: (a) collecting a blood sample from the patient; (b) detecting an inflammatory response in the patient when an amount of total mcfNA in the blood sample comprises mcfNA from at least two different microbes and exceeds a threshold amount of total mcfNA; and (c) administering an anti-inflammatory drug to the patient to treat the inflammatory response.

In yet another aspect, a method of treating an inflammatory response in a patient is provided, comprising: (a) collecting a blood sample from the patient; and (b) detecting an inflammatory response in the patient when an amount of total mcfNA in the blood sample comprises mcfNA from at least two different microbes and exceeds a threshold amount of total mcfNA.

In any of the preceding methods, in some embodiments, the subject has pneumonia. In any of the preceding methods, in some embodiments, the pneumonia is culture-positive pneumonia. In any of the preceding methods, in some embodiments, in some embodiments, the pneumonia is culture-negative pneumonia. In any of the preceding methods, in some embodiments, the mcfNA is mcfDNA. In any of the preceding methods, in some embodiments, the threshold amount of mcfNA is greater than 100,000 molecules per microliter of plasma (MPM). In any of the preceding methods, in some embodiments, the threshold amount of mcfNA is greater than 100,000 molecules per microliter of plasma (MPM) for mcfNA from known respiratory pathogens. In any of the preceding methods, in some embodiments, the method further comprises measuring levels of biomarkers of innate immunity or epithelial or endothelial injury in the plasma sample of the patient. In any of the preceding methods, in some embodiments, the biomarkers are selected from the group consisting of IL-6, IL-8, IL-10, RAGE, TNFR1, angiopoietin-2, procalcitonin, fractalkine, pentraxin-3, and ST2. In any of the preceding methods, in some embodiments, the biomarker is IL-8 or ST2. In any of the preceding methods, in some embodiments, the biomarker is procalcitonin or pentraxin-3. In any of the preceding methods, in some embodiments, the method further comprises comparing the amount of mcfNA in the subject with the biomarker levels using an algorithm to yield a test score. In any of the preceding methods, in some embodiments, the method further comprises administering a therapeutic drug to the subject based on the test score. In any of the preceding methods, in some embodiments, the subject is not bacteremic. In any of the preceding methods, in some embodiments, adapters are attached to the cell-free nucleic acids by ligation. In any of the preceding methods, in some embodiments, adapters are attached to the cell-free nucleic acids by primer extension. In any of the preceding methods, in some embodiments, the inflammatory response is a hyper-inflammatory response.

In yet another aspect, a method of detecting a bacterial infection in a patient with a COVID-19 infection is provided, comprising: (a) preparing a plasma sample from blood obtained from the patient with the COVID-19 infection, wherein the plasma sample comprises microbial cell-free nucleic acids (mcfNA); (b) producing a sequencing library comprising the mcfNA attached to the adapters; (c) conducting next generation sequencing on the sequencing library to produce sequence reads corresponding to the mcfNA; (d) aligning the sequence reads to sequences from a database comprising at least 1000 bacterial reference sequences; (e) determining an amount of mcfNA from at least one bacterium based on the aligning of the sequence reads; and (f) identifying a bacterial infection in the patient based on the amount of mcNA from the at least one bacterium.

In yet another aspect, a method of detecting a bacterial infection in a patient with a COVID-19 infection is provided, comprising: (a) preparing a plasma sample from blood obtained from the patient with the COVID-19 infection, wherein the plasma sample comprises microbial cell-free nucleic acids (mcfNA); (b) conducting next generation sequencing to produce sequence reads corresponding to the mcfNA; (c) aligning the sequence reads to sequences from a database comprising at least 1000 bacterial reference sequences; (d) determining an amount of mcfNA from at least one bacterium based on the aligning of the sequence reads; and (e) identifying a bacterial infection in the patient based on the amount of mcNA from the at least one bacterium.

In yet another aspect, a method of diagnosing and treating a bacterial infection in a patient with a COVID-19 infection is provided, comprising: (a) collecting a blood sample from the patient with the COVID-19 infection; (b) detecting the bacterial infection when an amount of bacterial mcfNA in the blood sample exceeds a threshold amount of mcfNA; and (c) administering a therapeutic drug to the patient to treat the bacterial infection.

In yet another aspect, a method of diagnosing and treating a bacterial infection in a patient with a COVID-19 infection is provided, comprising: (a) collecting a blood sample from the patient with the COVID-19 infection; and (b) detecting the bacterial infection when an amount of bacterial mcfNA in the blood sample exceeds a threshold amount of mcfNA.

In any of the preceding methods, in some embodiments, the patient has COVID-19 pneumonia. In any of the preceding methods, in some embodiments, wherein the bacterial infection is a respiratory infection. In any of the preceding methods, in some embodiments, the mcfNA (e.g., mcfDNA) is bacterial mcfNA from S. aureus, P. aeruginosa or K. pneumoniae. In some embodiments, the mcfNA (e.g., mcfDNA) is derived from at least one pathogen listed in Table 2. In some embodiments, the mcfNA (e.g., mcfDNA) is derived from at least one respiratory pathogen listed in Table 2. In any of the preceding methods, in some embodiments, the patient has culture-positive pneumonia. In any of the preceding methods, in some embodiments, the patient has culture-negative pneumonia. In any of the preceding methods, in some embodiments, the threshold amount of mcfNA is the amount of mcfNA measured in plasma of a healthy or uninfected subject. In any of the preceding methods, in some embodiments, the amount of mcfNA is measured by metagenomic next generation sequencing. In any of the preceding methods, in some embodiments, the mcfNA is mcfDNA. In any of the preceding methods, in some embodiments, the plasma is spiked with a known concentration of synthetic normalization controls.

In yet another aspect, a nucleic acid sequencing system for detecting secondary infection in a subject with a first infection is provided comprising: (a) a next-generation sequencing device comprising a flow cell and a computer processor that outputs data comprising sequence reads collected from measurements conducted in the flow cell; and (b) a computing device that comprises quantitation of total microbial cell-free nucleic acids (mcfNA) logic that (i) detects mcfNA from at least two different microbes by aligning the sequence reads to microbial reference sequence reads; (ii) calculates total mcfNA as a function of molecules per microliter of plasma, wherein the total mcfNA is an aggregate value of mcfNA from the at least two different microbes; and (iii) comprises an event generator to generate an event indicative a secondary infection when the total mcfNA exceeds a threshold value. In some embodiments, the quantitation of total microbial cell-free nucleic acids (mcfNA) logic comprises logic that excludes sequence reads from the analysis if they align to human reference sequences. In some embodiments, the quantitation of total microbial cell-free nucleic acids (mcfNA) logic comprises logic that excludes sequence reads from the analysis if they align to a synthetic nucleic acid reference. In some embodiments, the mcfNA is microbial cell-free DNA. In some embodiments, the threshold value is at least 600 MPM. In some embodiments, the threshold value is at least 4000 MPM.

In yet another aspect, a method of detecting secondary infection in a subject exhibiting pneumonia is provided, said method comprising (a) obtaining a plasma sample from said subject, (b) evaluating the amount of microbial cell-free nucleic acids in said sample; (c) comparing said amount of microbial cell free nucleic acids to a threshold level; and (d) detecting a secondary infection if said amount of microbial cell free nucleic acids exceeds said threshold level. In some embodiments, said subject has COVID-19. In some embodiments, said secondary infection is bacterial or fungal. In some embodiments, the method further comprises determining the presence and quantity of at least one bacterium, fungus or parasite in said subject.

In yet another aspect, a method of identifying a secondary infection at a site of localization in a subject with a viral infection is provided, comprising a) obtaining a plasma sample from said subject, (b) evaluating the amount of microbial cell-free nucleic acids in said sample; (c) comparing said amount of microbial cell free nucleic acids to a threshold level; and (d) detecting an infection at a site of localization in said subject if said amount of microbial cell free nucleic acids exceeds said threshold level. In some embodiments, said site of localization is the lungs.

In yet another aspect, anon-invasive method of detecting a respiratory infection in a subject exhibiting a pneumonia is provided, said method comprising a) obtaining a plasma sample from said subject, (b) evaluating the amount of microbial cell-free nucleic acids in said sample; (c) comparing said amount of microbial cell free nucleic acids to a threshold level; and (d) detecting a respiratory infection if said amount of microbial cell free nucleic acids exceeds said threshold level. In some embodiments, said subject has Covid-19 and is at risk for pneumonia.

In yet another aspect, a method for treating a patient suspected of having a secondary infection is provided, the method comprising: determining whether the patient will benefit from anti-microbial therapy by: determining in a sample from the patient a microbial cell-free nucleic acid level value (amount) and determining in a sample from the patient the level of a set of biomarkers, wherein the set of biomarkers comprises biomarkers of innate immunity (e.g., IL-8 and ST2) and/or bacterial infections (e.g., procalcitonin and pentraxin-3); and comparing the expression level values with the biomarker levels to yield a test score. In some embodiments, the method further comprises administering a treatment regimen comprising an anti-microbial therapy to the patient based on the test score.

In yet another aspect, a method for assessing the risk or prognosis of an inflammatory response in a subject with a disease is provided, the method comprising: performing at least one immunoassay on a blood sample from the subject to generate a first dataset comprising protein level data for at least two protein markers, wherein the at least two protein markers comprise at least two markers selected from fractalkine, interleukin(IL)-6, IL-8, pentraxin-3, procalcitonin, receptor for advanced glycation end products (RAGE), suppression of tumorigenicity (ST)-2, and tumour necrosis factor receptor (TNFR)-1 to provide a multi-biomarker inflammatory activity score (MBDA); performing at least one assay on a blood sample from the subject to generate determine the molecules per milliliter (MPM) of microbial cell-free DNA (mcfDNA); and determining the risk/prognosis of an elevated inflammatory response based on the mcfDNA MPM and MBDA score. In some embodiments, the disease is pulmonary pneumonia. In some embodiments, the subject has ventilator-associated pneumonia. In any of the preceding methods, in some embodiments, the inflammatory response is a hyper-inflammatory response.

In yet another aspect, a method of obtaining an inflammatory progression (IP) risk score for a subject with pneumonia is provided, said method comprising: obtaining or having obtained a biological sample from said subject; determining a multi-biomarker inflammatory activity score (MBDA) for said subject; determining the molecules per milliliter (MPM) of microbial cell-free DNA (mcfDNA); and obtaining an IP risk score from said subject's MBDA and MPM using an interpretation function. In some embodiments, the inflammatory response is a hyper-inflammatory response.

In yet another aspect, a method of detecting a localized respiratory infection in a subject is provided, the method comprising: obtaining or providing a plasma sample from the subject, wherein the subject is not bacteremic and the plasma sample comprises cell-free nucleic acids; performing next generation sequencing or metagenomic sequencing on cell-free nucleic acids from the plasma sample and producing sequence reads; and aligning the sequence reads with sequences of respiratory pathogens in order to detect the presence and quantity of at least one respiratory pathogen, wherein the at least one respiratory pathogen is associated with the localized respiratory infection. In some embodiments, the cell-free nucleic acids are cell-free DNA. In some embodiments, the sequence reads aligned with the sequences of respiratory pathogens correspond to microbial cell-free DNA. In some embodiments, the respiratory infection is pneumonia. In some embodiments, the respiratory infection is bacterial pneumonia. In some embodiments, the at least one respiratory pathogen is at least one bacterium associated with a respiratory infection. In some embodiments, the respiratory infection is a bacterial respiratory infection. In some embodiments, the at least one respiratory pathogen is S. aureus, P. aeruginosa or K. pneumoniae. In some embodiments, the at least one respiratory pathogen is at least one respiratory pathogen listed in Table 2. In some embodiments, the method further comprises adding synthetic nucleic acids to the plasma sample. In some embodiments, the method further comprises performing next generation sequencing on the synthetic nucleic acids. In some embodiments, the synthetic nucleic acids are normalization controls. In some embodiments, the method further comprises attaching adapters to the cell-free nucleic acids in order to produce cell-free nucleic acids attached to the adapters. In some embodiments, the adapters are ligated to the cell-free nucleic acids. In some embodiments, the adapters are attached to the cell-free nucleic acids by a primer extension reaction. In some embodiments, the adapters comprise a sequence unique to the subject. In some embodiments, the method further comprises combining the cell-free nucleic acids attached to the adapters with cell-free nucleic acids obtained from a different subject. In some embodiments, the cell-free nucleic acids obtained from a different subject are attached to adapters that comprise a sequence unique to the different subject. In some embodiments, the method further comprises administering a treatment (e.g., antibiotic) to the subject to treat the respiratory infection. In some embodiments, the method further comprises administering an antibiotic to treat the at least one pathogen associated with the respiratory infection. In some cases, the subject is blood culture negative. In some embodiments, the subject is blood culture positive. In some embodiments, culture of secretions from the respiratory tract is positive. In some embodiments, culture of the respiratory tract secretions is negative. In some embodiments, the subject has bacterial pneumonia and a viral pneumonia. In some cases, the viral pneumonia is caused by SARS-CoV-2 virus. In some embodiments, the bacterial pneumonia is caused by S. aureus, P. aeruginosa or K. pneumoniae. In some embodiments, the bacterial pneumonia is caused by a respiratory pathogen listed in Table 2.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

FIG. 1A shows total mcfDNA (MPM) for patients with culture-positive pneumonia, uninfected controls, culture-negative pneumonia, and COVID-19. The mean values are shown with a horizontal bar, the standard deviation by rectangles. Statistical significance (asterisks) is shown for culture-positive pneumonia vs. COVID-19 (p<0.001), and uninfected controls vs. COVID-19 (p<0.05). FIG. 1B shows the regression co-efficient (95% CI) and p-values of biomarkers associated with different pathways.

FIG. 2A and FIG. 2B show non-survivors of severe COVID-19 infection had higher microbial cell-free DNA molecules per microliter of plasma by metagenomic sequencing compared to survivors(median [interquartile range]: 11,125 [650-26,436] vs. 661 [1], Wilcoxon test p-value=0.04) and a trend for higher number of identified microbes per sample (3.5 [1.8-4.3] vs. 1.0 [0-2.5], Wilcoxon test p-value=0.06). FIG. 2A shows total mcfDNA molecules per microliter. FIG. 2B shows N of microbes detected by plasma metagenomics.

FIG. 3 shows case-based analysis of 15 critically ill patients with COVID-19 with depicted clinical diagnoses, plasma microbial cell-free DNA metagenomics and survival outcomes. The Y-axis margin indicates two groups of clinical diagnoses: Group A includes eleven patients who received antibiotics for either microbiologically confirmed (n=3) or clinically suspected infections despite negative microbiologic workup (n=8), whereas Group B includes four patients with low clinical suspicion for secondary infection and no antibiotic therapies at time of sampling. The Y-axis ticks denote each patient sample, and the x-height of each stacked bar represents the number of microbial cell-free DNA molecules per plasma microliter (MPMs) by metagenomic sequencing, with different colors for the top ten microbes by ranked abundance. The “other” category (shown in grey) represents the sum of lower abundance taxa of commensal origin. Five out of eleven subjects of Group A (45%, Subjects 1-5) had high MPM signal for probable respiratory pathogens, whereas in the remaining 6/11 subjects there was no evidence of co-infecting bacterial pathogens. Subject 7 was clinically-diagnosed with culture-negative sepsis and treated with prolonged course of empiric broad-spectrum antibiotics while on extracorporeal membrane oxygenation support for refractory hypoxemic respiratory failure from COVID-19; the high mcfDNA signal for C. tropicalis (2,490 MPMs) is concerning for undiagnosed invasive Candidiasis, corroborated by persistent growth of yeast organisms (not further speciated) from clinical bronchoalveolar lavage samples obtained on days 5, 9 and 14 after the research sample acquisition. Two out of four patients of Group B (subjects 12 and 13) who did not survive and had not received empiric antimicrobials were found to have high mcfDNA signal (>4000 total MPMs) of probable respiratory pathogens, indicative of undiagnosed (and untreated) secondary infections.

FIG. 4A shows plasma microbial cell-free DNA levels are elevated in culture-positive pneumonia compared with culture-negative pneumonia and uninfected controls and compared to culture-negative pneumonia patients (pairwise comparisons post hoc adjusted by Benjamini-Hochberg method). *, post hoc p<0.05; ***, post hoc p<0.005; ****, post hoc p<0.001. FIG. 4B shows the types of mcfDNA (bacterial, fungal, or viral) detected in culture-positive, culture-negative pneumonia and in uninfected controls depicted in pie charts. The radius of pie charts scales quadratically proportional to the sum of mcfDNA MPMs detected within each patient subgroup. The proportion of viral mcfDNA was significantly higher in the culture-negative (18.0%) compared to the culture-positive pneumonia (1.6%) group (p<0.0001 for z test of comparison of proportions).

FIG. 5A and FIG. 5B show circulating mcfDNA is associated with host inflammatory responses in patients with pneumonia. FIG. 5A is a graphical representation of linear regression models of plasma biomarkers (outcomes, shown in y-axis) against plasma mcfDNA levels (predictor, shown in x-axis) in unadjusted as well as adjusted models for a priori selected potential confounders, including (i) a surrogate of the microbial inoculum (culture-positive vs. negative classification), (ii) degree of lung injury (as depicted radiographically by RSI and by the epithelial injury biomarker receptor for advanced glycation end products-RAGE), and (iii) host innate immunity status (age, chronic obstructive pulmonary disease and immunosuppression). The direction of the effect size and corresponding statistical significance for the regression coefficient of mcfDNA on each plasma biomarker are visually presented by color and size coding, respectively; regression results are listed in detail in Table 4. FIG. 5B is a graph of host-response sub-phenotypes. Patients with pneumonia assigned to the hyperinflammatory sub-phenotype had significantly higher mcfDNA compared to hypo-inflammatory patients (median 7,731, interquartile range-IQR, MPMs, [3,100-79,849] vs. 546 [0-4,609] respectively, p<0.05). We assigned patients to the hyper- vs. hypo-inflammatory sub-phenotype based on a parsimonious predictive model utilizing levels of angiopoietin-2, procalcitonin, TNFR1 and bicarbonate.

FIG. 6A and FIG. 6B show the impact of timing of sampling and antibiotic exposure on mcfDNA and procalcitonin levels in patients with pneumonia. FIG. 6A shows time of sampling from ICU admission between culture positive and culture negative patients. FIG. 6B shows time of sampling from intubation between culture positive and culture negative patients. Culture-positive patients had relatively shorter time interval from intubation compared to culture-negative patients (p=0.014, Wilcoxon test). FIG. 6C and FIG. 6D shows procalcitonin levels did not differ by time of sampling from ICU admission (FIG. 6D) or intubation (FIG. 6C). FIG. 6E and FIG. F shows mcfDNA levels did not differ by time of sampling from ICU admission (FIG. 6F) or intubation (FIG. 6E). FIG. 6G and FIG. 6H shows procalcitonin (FIG. 6G) and mcfDNA levels (FIG. 6H) were not significantly associated with the antibiotic exposure score, applied as previously described. Kitsios 2020; Zhao, 2014, Sci Rep, 4:4345.

FIG. 7A and FIG. 7B illustrate that the mcfDNA of recognized respiratory pathogens was significantly associated with clinical diagnosis of pneumonia and inflammatory biomarker levels. Direction of the effect size and corresponding statistical significance for the regression coefficient of mcfDNA on each plasma biomarker are visually presented by color and size coding, respectively. Abbreviations: Ang-2, angiopoietin-2; IL, interleukin; RAGE, receptor for advanced glycation product; ST-2, suppression of tumorigenicity-2; TNFR-1, tumor necrosis factor receptor 1.

FIG. 8A and FIG. 8B show the sum of mcfDNA load detected across all participants by taxa, quantified as molecules per microliter (MPMs). FIG. 8A shows mcfDNA of recognized respiratory pathogen taxa; FIG. 8B shows mcfDNA of microbes with unclear clinical importance.

### DETAILED DESCRIPTION

The invention will now be described in detail by way of reference only using the following definitions and examples. All patents and publications, including all sequences disclosed within such patents and publications, referred to herein are expressly incorporated by reference in their entireties.

### Overview

Provided herein are methods, devices, and systems for analyzing total microbial cell-free nucleic acids, particularly total microbial cell-free DNA (“total mcfDNA”), in order to detect or predict or otherwise evaluate a secondary infection in a subject, a hyperinflammatory response in a subject, or severity of infection in a subject. In some cases, the total microbial cell-free nucleic acids (e.g., total mcfDNA) is used to detect or predict or otherwise evaluate whether a patient (e.g., a patient with COVID-19) is likely to survive. Often, the subject is culture-negative for bacteria or viral pathogens that can cause the secondary infection or hyperinflammatory response at the time a sample is collected from the patient. The samples used in this disclosure are generally plasma samples or other samples that can be obtained relatively non-invasively. In some embodiments, the subject has pneumonia. In some cases, the subject has culture-positive pneumonia. In some cases, the subject has culture-negative pneumonia. In some cases, the subject has a COVID-19 infection. In some cases, the subject has COVID-19 pneumonia or severe COVID-19. In some cases, the threshold value for total microbial cell-free nucleic acids (e.g., mcfDNA) is an aggregate value for mcfNA (e.g., mcfDNA) from at least two different microbes. In some embodiments, the threshold value for total mcfNA (e.g., total mcfDNA) is 400 molecules per microliter of plasma (MPM), 600 MPM, 1000 MPM, 5000 MPM, 10000 MPM, or 100000 MPM. In some cases, the total mcfDNA reflects the total mcfDNA that derives from bacterial microbes. In some cases, the total mcfDNA reflects the total mcfDNA that derives from respiratory pathogens. In some embodiments, the respiratory pathogen is at least one respiratory pathogen listed in Table 2, in any combination. In some embodiments, the respiratory pathogen is a streptococcus, pseudomonas, or klebsiella bacterium. In some embodiments, the respiratory pathogen is from any genus listed in Table 2. In some cases, the respiratory pathogen is from the genus Actinomyces, Aspergillus, Bacteroides, Citrobacter, Cytomegalovirus, Enterobacter, Eschericihia, Enterococcus, Streptooccus, Pseudomonas, Klebsiella, and/or Haemophilus, In some cases, the respiratory pathogen is S. aureus, P. aeruginosa and/or K. pneumoniae, in any combination.

In some cases, the method comprises detecting a secondary infection in a patient with COVID-19, wherein the method comprises detecting at least one microbe associated with the secondary infection by performing next generation sequencing (e.g., metagenomic next generation sequencing) on microbial cell-free nucleic acids (e.g., microbial cell-free DNA (mcfDNA)) obtained from a sample (e.g., plasma) obtained from the subject. In some cases, the secondary infection is a bacterial infection and the COVID-19 patient is culture negative for the bacterial infection. In some cases, the secondary infection is a bacterial infection that is caused by a respiratory microbe (e.g., a bacterium that causes a respiratory infection or pneumonia). In some cases, the secondary infection is a bacterial pneumonia infection.

The methods provided herein have multiple uses and advantages. For example, the methods provide reliable methods for detecting a secondary infection in a patient, particularly when the secondary infection is not detectable by culture. The methods can also help identify the causative agents of a secondary pneumonia in patients with COVID-19 pneumonia, particularly when clinical distinction between the secondary pneumonia and COVID-19 pneumonia is challenging, or even not possible. The methods provide the further advantage of detecting pathogens associated with secondary pneumonia even when the patient has been administered an antibiotic, which can, in some cases, limit the sensitivity of microbiologic studies. The non-invasive nature of the methods provided herein also has the advantage of avoiding subjecting a patient to the discomfort and risks associated with bronchoscopy, as well as limiting exposure of healthcare personnel to SARS-COV-2 that is potentially aerosolized during a bronchoscopy procedure.

The headings provided herein are not limitations of the various aspects or embodiments of the invention which can be had by reference to the specification. Accordingly, the terms defined immediately below are more fully defined by reference to the specification.

All definitions herein described whether specifically mentioned or not, should be construed to refer to definitions as used throughout the specification and attached claims.

In the present disclosure, wherever aspects are described herein with the language “comprising,” otherwise analogous aspects described in terms of “consisting of” and/or “consisting essentially of” are also provided.

Numeric ranges are inclusive of the numbers defining the range. The term “about” as used herein generally means plus or minus ten percent (10%) of a value, inclusive of the value, unless otherwise indicated by the context of the usage. For example, “about 100” refers to any number from 90 to 110, inclusive of 100.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” “at most,” or “less than or equal to” precedes the first numerical value in a series of two or more numerical values, the term “no more than,” “at most,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Unless otherwise indicated, nucleic acids are written left to right in 5′ to 3′ orientation; amino acid sequences are written left to right in amino to carboxy orientation, respectively.

The term “attach” and its grammatical equivalents may refer to connecting two molecules using any mode of attachment. For example, attaching may refer to connecting two molecules by chemical bonds or other method to generate a new molecule. Attaching an adapter to a nucleic acid may refer to forming a chemical bond between the adapter and the nucleic acid. In some cases, attaching is performed by ligation, e.g., using a ligase. For example, a nucleic acid adapter may be attached to a target nucleic acid by ligation, via forming a phosphodiester bond catalyzed by a ligase. In some embodiments, the attachment comprises attaching via performing a primer extension reaction, wherein the sequence to be attached is present in the primer.

As used herein, the term “or” is used to refer to a nonexclusive or, such as “A or B” includes “A but not B,” “B but not A,” and “A and B,” unless otherwise indicated.

As used herein, “a”, “an”, and “the” can include plural referents unless otherwise limited expressly or by context.

“Interpretation function,” as used herein, means the transformation of a set of observed data into a meaningful determination of particular interest; e.g., an interpretation function may be a predictive model that is created by utilizing one or more statistical algorithms to transform a dataset of observed biomarker data and/or MPM into a meaningful determination of disease activity or the disease state of a subject.

By a “multi-biomarker disease activity score”, “multi-biomarker disease activity index score”, “MBDA score” or simply “MBDA” is intended a score that provides a semi-quantitative measure of inflammatory disease activity or the state of inflammatory disease in a subject. The interpretation function, in some embodiments, can be created from predictive or multivariate modeling based on statistical algorithms. In some embodiments, input to the interpretation function can comprise the results of testing one or more, two or more, three or more, four or more, five or more, six or more, seven or more, eight or more, nine or more, ten or more, 11 or more, 15 or more, 20 or more, 50 or more, or 100 or more biomarkers alone or in combination with microbial cell-free DNA measurements, also described herein. In some embodiments, the MBDA score is an indirect measure of inflammatory disease activity. In some embodiments, the MBDA score is a quantitative measure of inflammatory disease activity.

In some embodiments, the interpretation function is based on a predictive model. Established statistical algorithms and methods, useful as models or useful in designing predictive models, can include but are not limited to: analysis of variants (ANOVA); Bayesian networks; boosting and Ada-boosting; bootstrap aggregating (or bagging) algorithms; decision trees classification techniques, such as Classification and Regression Trees (CART), boosted CART, Random Forest (RF), Recursive Partitioning Trees (RPART), and others; Curds and Whey (CW); Curds and Whey-Lasso; dimension reduction methods, such as principal component analysis (PCA) and factor rotation or factor analysis; discriminant analysis, including Linear Discriminant Analysis (LDA), Eigengene Linear Discriminant Analysis (ELDA), and quadratic discriminant analysis; Discriminant Function Analysis (DFA); factor rotation or factor analysis; genetic algorithms; Hidden Markov Models; kernel based machine algorithms such as kernel density estimation, kernel partial least squares algorithms, kernel matching pursuit algorithms, kernel Fisher's discriminate analysis algorithms, and kernel principal components analysis algorithms; linear regression and generalized linear models, including or utilizing Forward Linear Stepwise Regression, Lasso (or LASSO) shrinkage and selection method, and Elastic Net regularization and selection method; glmnet (Lasso and Elastic Net-regularized generalized linear model); Logistic Regression (LogReg); meta-learner algorithms; nearest neighbor methods for classification or regression, e.g. Kth-nearest neighbor (KNN); non-linear regression or classification algorithms; neural networks; partial least square; rules based classifiers; shrunken centroids (SC); sliced inverse regression; Standard for the Exchange of Product model data, Application Interpreted Constructs (StepAIC); super principal component (SPC) regression; and, Support Vector Machines (SVM) and Recursive Support Vector Machines (RSVM), among others. Additionally, clustering algorithms as are known in the art can be useful in determining subject sub-groups.

Logistic Regression is the traditional predictive modeling method of choice for dichotomous response variables; e.g., treatment 1 versus treatment 2. It can be used to model both linear and non-linear aspects of the data variables and provides easily interpretable odds ratios.

Discriminant Function Analysis (DFA) uses a set of analytes as variables (roots) to discriminate between two or more naturally occurring groups. DFA is used to test analytes that are significantly different between groups. A forward stepwise DFA can be used to select a set of analytes that maximally discriminate among the groups studied. Specifically, at each step all variables can be reviewed to determine which will maximally discriminate among groups. This information is then included in a discriminative function, denoted a root, which is an equation consisting of linear combinations of analyte concentrations for the prediction of group membership. The discriminatory potential of the final equation can be observed as a line plot of the root values obtained for each group. This approach identifies groups of analytes whose changes in concentration levels can be used to delineate profiles, diagnose and assess therapeutic efficacy. The DFA model can also create an arbitrary score by which new subjects can be classified as either “healthy” or “diseased.” To facilitate the use of this score for the medical community the score can be rescaled so a value of 0 indicates a healthy individual and scores greater than 0 indicate increasing risk.

Classification and regression trees (CART) perform logical splits (if/then) of data to create a decision tree. All observations that fall in each node are classified according to the most common outcome in that node. CART results are easily interpretable—one follows a series of if/then tree branches until a classification results.

Support vector machines (SVM) classify objects into two or more classes. Examples of classes include sets of treatment alternatives, sets of diagnostic alternatives, or sets of prognostic alternatives. Each object is assigned to a class based on its similarity to (or distance from) objects in the training data set in which the correct class assignment of each object is known. The measure of similarity of a new object to the known objects is determined using support vectors, which define a region in a potentially high dimensional space (>R6).

The process of bootstrap aggregating, or “bagging,” is computationally simple. In the first step, a given dataset is randomly resampled a specified number of times (e.g., thousands), effectively providing that number of new datasets, which are referred to as “bootstrapped resamples” of data, each of which can then be used to build a model. Then, in the example of classification models, the class of every new observation is predicted by the number of classification models created in the first step. The final class decision is based upon a “majority vote” of the classification models; i.e., a final classification call is determined by counting the number of times a new observation is classified into a given group and taking the majority classification (33%+ for a three-class system). In the example of logistical regression models, if a logistical regression is bagged 1000 times, there will be 1000 logistical models, and each will provide the probability of a sample belonging to class 1 or 2.

Curds and Whey (CW) using ordinary least squares (OLS) is another predictive modeling method. Breiman, 1997, J. Royal. Stat. Soc. B, 59:3-54. This method takes advantage of the correlations between response variables to improve predictive accuracy, compared with the usual procedure of performing an individual regression of each response variable on the common set of predictor variables X. In CW, Y=XB*S, where Y=(ykj) with k for the kth patient and j for jth response (j=1 for TJC, j=2 for SIC, etc.), B is obtained using OLS, and S is the shrinkage matrix computed from the canonical coordinate system. Another method is Curds and Whey and Lasso in combination (CW-Lasso). Instead of using OLS to obtain B, as in CW, here Lasso is used, and parameters are adjusted accordingly for the Lasso approach.

Many of these techniques are useful either combined with a biomarker selection technique (such as, for example, forward selection, backwards selection, or stepwise selection), or for complete enumeration of all potential panels of a given size, or genetic algorithms, or they can themselves include biomarker selection methodologies in their own techniques. These techniques can be coupled with information criteria, such as Akaike's Information Criterion (AIC), Bayes Information Criterion (BIC), or cross-validation, to quantify the tradeoff between the inclusion of additional biomarkers and model improvement, and to minimize overfit. The resulting predictive models can be validated in other studies, or cross-validated in the study they were originally trained in, using such techniques as, for example, Leave-One-Out (LOO) and 10-Fold cross-validation (10-Fold CV).

By “prognosis” is intended a prediction as to the likely outcome of a disease. Prognostic estimates are useful in, among other things, determining an appropriate therapeutic regimen for a subject.

A “multiplex assay” as used herein refers to an assay that simultaneously measures multiple analytes, e.g., multiple nucleic acid analytes, multiple DNA analytes, multiple cell-free DNA analytes, multiple protein analytes, in a single run or cycle of the assay.

A “predictive model,” which term may be used synonymously herein with “multivariate model” or simply a “model,” is a mathematical construct developed using a statistical algorithm or algorithms for classifying sets of data. The term “predicting” refers to generating a value for a datapoint without actually performing the clinical diagnostic procedures normally or otherwise required to produce that datapoint; “predicting” as used in this modeling context should not be understood solely to refer to the power of a model to predict a particular outcome. Predictive models can provide an interpretation function; e.g., a predictive model can be created by utilizing one or more statistical algorithms or methods to transform a dataset of observed data into a meaningful determination of a risk score or the disease state of a subject.

A “quantitative dataset” or “quantitative data” as used in the present teachings, refers to the data derived from, e.g., detection and composite measurements of expression of a plurality of biomarkers (i.e., two or more) in a subject sample. The quantitative dataset can be used to generate a score for the identification, monitoring and treatment of disease states, and in characterizing the biological condition of a subject. It is possible that different biomarkers will be detected depending on the disease state or physiological condition of interest.

“Biomarker,” “biomarkers,” “marker” or “markers” in the context of the present disclosure encompasses, without limitation, cytokines, chemokines, growth factors, proteins, peptides, nucleic acids, oligonucleotides, and metabolites, together with their related metabolites, mutations, isoforms, variants, polymorphisms, modifications, fragments, subunits, degradation products, elements, and other analytes or sample-derived measures. Biomarkers can also include mutated proteins, mutated nucleic acids, variations in copy numbers and/or transcript variants. Biomarkers also encompass non-blood borne factors and non-analyte physiological markers of health status, and/or other factors or markers not measured from samples (e.g., biological samples such as bodily fluids), such as clinical parameters and traditional factors for clinical assessments. Biomarkers can also include any indices that are calculated and/or created mathematically.

Biomarkers can also include combinations of any one or more of the foregoing measurements, including temporal trends and differences. In some embodiments, biomarkers are two or more of the following: fractalkine, interleukin-8, procalcitonin, pentraxin-3, suppression of tumorigenicity-2 (ST-2), and soluble tumor necrosis factor receptor-1 (TNFR-1). In some embodiments, biomarkers are one or more, two or more, three or more, four or more, five or more, or six of the following: fractalkine, interleukin-8, procalcitonin, pentraxin-3, suppression of tumorigenicity-2 (ST-2), and soluble tumor necrosis factor receptor-1 (TNFR-1).

Subjects

By “subject” is generally intended a mammal, particularly a human, such as a human patient. The term “mammal” includes but is not limited to a human, non-human primate, dog, cat, mouse, rat, cow, horse, pig, sheep, and camel. Mammals other than humans can be advantageously used as subjects that represent animal models of inflammation or secondary infection. A subject may be male, female, adult, immature, or young.

In some embodiments, the subject has a first infection, e.g., viral infection, COVID-19 infection, pneumonia, viral pneumonia, culture-positive infection, culture-negative infection, culture-positive pneumonia, culture-negative pneumonia. A subject may be one who has been previously diagnosed or identified as having an inflammatory disease. A subject can be one who has already undergone or is undergoing a therapeutic intervention for an inflammatory disease. A subject may also be one who has not been previously diagnosed as having an inflammatory disease; for example a subject may be one who exhibits one or more symptoms or risks factors for an inflammatory condition, or a subject who does not exhibit symptoms or risk factors for an inflammatory condition, or a subject who is asymptomatic for inflammatory disease. In some cases, the inflammatory condition is a hyper-inflammatory response.

Identifying the risk of inflammatory progression (IP) in a subject can allow for a prognosis of the disease and thus for the informed selection of, initiation of, adjustment of or increasing or decreasing various therapeutic regimens to delay, reduce or prevent that subject's progression to a more advanced disease state, e.g. a hyperinflammatory response. Subjects can be identified as having a particular risk of IP and so can be selected to begin or accelerate treatment to prevent or delay the further progression of inflammatory disease. In some cases, subjects can be identified as having a low or moderate risk of IP, and so can be selected to have their treatment decreased or discontinued. In other embodiments subjects may be identified by their IP risk scores as being at a particular risk for IP and can have therapy selected based on IP risk.

In some embodiments, the subject has, is suspected of having, or is at risk of having an infection by a bacterium, a fungus, a virus, a parasite, or any combination thereof. Such infection can be a secondary infection, such as an infection secondary to viral pneumonia, COVID-19 infection, viral infection, COVID-19 pneumonia, or other first infection. In some embodiments, an infection by a bacteria, a fungus, a virus, a parasite, or any combination thereof is a respiratory infection, e.g., pneumonia. In some embodiments, the infection is a fungal infection. In some embodiments, the infection is a bacterial infection. In some embodiments, a bacterial or fungal infection can comprise an infection by an organism selected from the group consisting of Bacillus spp., Clostridium spp, Corynebactehum jeikeium, Enterococcus spp., Lactobacillus spp., Rothia spp., Staphylococcus spp., Streptococcus spp., Citrobacter spp., Escherichia coli, Klebsiella spp., Pseudomonas spp., Stenotrophomonas maltophilia, and Candida spp. In some embodiments, the bacterial infection is a gram-negative bacterial infection. In some embodiments, the bacterial infection is a gram-positive bacterial infection, In some embodiments, the bacterial or fungal infection is susceptible to empirical antimicrobial therapy. In some embodiments, a subject is diagnosed with having an infection or with having a hyper-inflammatory response using methods disclosed herein. In some embodiments, a subject is diagnosed with having an increased risk of having severe disease or increased risk of death from the infection. For example, in some embodiments, the methods can detect that the subject has an increased risk of severe COVID-19, risk of a hyper-inflammatory response, and/or heightened risk of death from COVID-19.

In some cases, the subject has a localized infection. In some embodiments, the localized infection is a localized lung infection, e.g., pneumonia. In some cases, the subject is not bacteremic. In some cases, mcfDNA derived from a pathogen (e.g., respiratory pathogen) is detected in the subject, in the absence of bacteremia. In some cases, such mcfDNA is detected in plasma of a subject. For example, in some cases, the methods provided herein allow for detection in a plasma sample of a mcfDNA derived from a respiratory pathogen (e.g., bacterial pathogen associated with a respiratory infection) in a subject with a localized infection (e.g., pneumonia) and who does not have bacteremia.

Samples

A “sample” in the context of the present disclosure refers to any biological sample that is isolated from a subject. A sample can include, without limitation, a single cell or multiple cells, fragments of cells, an aliquot of body fluid, whole blood, platelets, serum, plasma, red blood cells, white blood cells or leucocytes, endothelial cells, tissue biopsies, synovial fluid, lymphatic fluid, ascites fluid, or interstitial or extracellular fluid. The term “sample” also encompasses the fluid in spaces between or external to the tissues that produce them, including synovial fluid, gingival crevicular fluid, bone marrow, cerebrospinal fluid (CSF), saliva, mucous, sputum, semen, sweat, urine or bodily fluids generally. “Blood sample” can refer to whole blood or any fraction thereof, including but not limited to blood cells, red blood cells, white blood cells, platelets, serum and plasma. Samples can be obtained from a subject by any means known in the art including, but not limited to, venipuncture, excretion, biopsy, needle aspirate, lavage, scraping, surgical incision or intervention or other methods known in the art.

In some embodiments, a sample is collected from a subject (e.g., a patient). Samples can be obtained from a subject by any methods known in the art including, but not limited to, venipuncture, excretion, biopsy, needle aspirate, lavage, scraping,

In some embodiments, a sample is a biological sample. In some embodiments, the biological sample is a whole blood sample. In some embodiments, the sample is a cell-free sample, such as a plasma sample or a cell-free plasma sample. In some embodiments, the sample is a sample of isolated or extracted nucleic acids (e.g., DNA, RNA, cell-free DNA). In some embodiments, the plasma sample is collected by collecting blood through venipuncture. In some embodiments, a specimen is mixed with an additive immediately after collection. In some cases, the additive is an anti-coagulant. In some cases, the additive prevents degradation of nucleic acids. In some cases, the additive is EDTA. In some embodiments, measures can be taken to avoid hemolysis or lipemia. In some embodiments, a sample is processed or unprocessed. In some embodiments, a sample is processed by extracting nucleic acids from a biological sample. In some embodiments, DNA is extracted from a sample. In some embodiments, nucleic acids are not extracted from the sample. In some embodiments, a sample comprises nucleic acids. In some embodiments, a sample consists essentially of nucleic acids.

In some cases, the methods provided herein comprise processing whole blood into a plasma sample. In some embodiments, such processing comprises centrifuging the whole blood in order to separate the plasma from blood cells. In some cases, the method further comprises subjecting the plasma to a second centrifugation, often at a higher speed in order to remove bacterial cells and cellular debris. In some cases, the second centrifugation is at a relative centrifugal force (rcf) of least about 4,000 rcf, at least about 5,000 rcf, at least about 6,000 rcf, at least about 8,000 rcf, at least about 10,000 rcf, at least about 12,000 rcf, at least about 14,000 rcf, at least about 16,000 rcf, or at least about 20,000 rcf.

At time of collection of a sample from the subject, the subject can be culture-negative for a microbe that is subsequently detected by a method provided herein. In some embodiments, at time of collection of a sample from the subject, the subject is culture-negative for a microbe that is subsequently detected by a method provided herein and the subject later becomes culture-positive for the microbe at a point in time following the collection of the sample. In some cases, at time of collection of the sample from the subject, the subject is culture-positive for a microbe that is subsequently detected by a method provided herein.

Often, a sample disclosed herein comprises a target nucleic acid (e.g., target DNA, target RNA). In some embodiments, a target nucleic acid is a cell-free nucleic acid or circulating cell-free nucleic acid. For example, the sample can comprise microbial cell-free nucleic acids (e.g., mcfDNA) that comprises a microbial target DNA (e.g., mcfDNA derived from a microbe, which can include pathogenic microbes). Exemplary microbes that can be detected by the methods provided herein include bacteria, fungi, parasites, and viruses. In some embodiments, a cell-free nucleic acid is a circulating cell-free nucleic acid. In some embodiments, a cell free nucleic acid can comprise cell-free DNA.

In some embodiments, nucleic acids (e.g., cell-free nucleic acids, cell-free DNA, RNA, or other nucleic acid in any combination thereof) are extracted from a sample. In some embodiments, isolated nucleic acids (e.g., extracted DNA) can be used to prepare DNA libraries. In some embodiments, DNA libraries can be prepared by attaching adapters to nucleic acids. In some embodiments, adapters can be used for sequencing of nucleic acids. In some embodiments, nucleic acids can comprise DNA. In some embodiments, nucleic acids containing adapters can be sequenced to obtain sequence reads. In some embodiments, a sample (e.g., a plasma sample comprising mcfDNA) is mixed with adapters prior to extracting nucleic acids or DNA from the sample. In some embodiments, nucleic acids extracted from a sample (e.g., a plasma sample comprising mcfDNA) are attached to adapters following extraction. In some embodiments, sequence reads can be produced through high-throughput sequencing (HTS). In some embodiments, HTS can comprise next-generation sequencing (NGS). In some cases, the HTS is metagenomic sequencing or metagenomic next generation sequencing. In some embodiments, sequence reads can be aligned to sequences in a reference dataset. In some cases, the reference dataset has sequences from at least 2, 5, 7, 10, 50, 100, 500, 750, 800, 900, 1000, or 2000 different microbes (e.g., bacteria, viruses, parasites, fungi). In some embodiments, the sequences are derived from a combination of respiratory pathogens, particularly bacteria associate with respiratory infections. In some embodiments, sequences can be a bacterial sequence aligned to a reference dataset to obtain an aligned sequence read. In some embodiments, a sequence can be a fungal sequence aligned to a reference dataset to obtain an aligned sequence read. In some embodiments, an aligned bacterial sequence, a fungal sequence or a combination thereof, can be quantified for bacterial sequences or fungal sequences based on aligned sequence reads obtained.

In the methods provided herein, nucleic acids can be isolated, extracted or purified. In some embodiments, nucleic acids can be extracted using a liquid extraction. In some embodiments, a liquid extraction can comprise a phenol-chloroform extraction. In some embodiments, a phenol-chloroform extraction can comprise use of Trizol™, DNAzol™, or any combination thereof. In some embodiments, nucleic acids can be extracted using centrifugation through selective filters in a column. In some embodiments, nucleic acids can be concentrated or precipitated by known methods, including, by way of example only, centrifugation. In some embodiments, nucleic acids can be bound to a selective membrane (e.g., silica) for the purposes of purification. In some embodiments, nucleic acids can be extracted using commercially available kits (e.g., QIAamp Circulating Nucleic Acid Kit™, Qiagen DNeasy kit™, QIAamp kit™, Qiagen Midi kit™, QIAprep spin kit™, or any combination thereof). Nucleic acids can also be enriched for fragments of a desired length, e.g., fragments which are less than 1000, 500, 400, 300, 200 or 100 base pairs in length. In some embodiments, enrichment based on size can be performed using, e.g., PEG-induced precipitation, an electrophoretic gel or chromatography material (Huber et al. (1993) Nucleic Acids Res. 21:1061-6), gel filtration chromatography, or TSKgel (Kato et al. (1984) J. Biochem, 95:83-86), which publications are hereby incorporated by reference in their entireties for all purposes.

In some embodiments, a nucleic acid sample is enriched for a target nucleic acid. In some embodiments, a target nucleic acid is a microbial cell-free nucleic.

In some embodiments, target (e.g., pathogen, microbial) nucleic acids is enriched relative to background (e.g., subject) nucleic acids in a sample, for example, by pull-down (e.g., preferentially pulling down target nucleic acids in a pull-down assay by hybridizing them to complementary oligonucleotides conjugated to a label such as a biotin tag and using, for example, avidin or streptavidin attached to a solid support), targeted PCR, or other methods. Examples of enrichment techniques include, but are not limited to: (a) self-hybridization techniques in which a major population in a sample of nucleic acids self-hybridizes more rapidly than a minor population in a sample; (b) depletion of nucleosome-associated DNA from free DNA; (c) removing and/or isolating DNA of specific length intervals; (d) exosome depletion or enrichment; and (e) strategic capture of regions of interest.

In some embodiments, an enriching step can comprise preferentially removing nucleic acids from a sample that are above about 120, about 150, about 200, or about 250 bases in length. In some embodiments, an enriching step comprises preferentially enriching nucleic acids from a sample that are between about 10 bases and about 60 bases in length, between about 10 bases and about 120 bases in length, between about 10 bases and about 150 bases in length, between about 10 bases and about 300 bases in length between about 30 bases and about 60 bases in length, between about 30 bases and about 120 bases in length, between about 30 bases and about 150 bases in length, between about 30 bases and about 200 bases in length, or between about 30 bases and about 300 bases in length. In some embodiments, an enriching step comprises preferentially digesting nucleic acids derived from the host (e.g., subject). In some embodiments, an enriching step comprises preferentially replicating the non-host nucleic acids.

In some embodiments, a nucleic acid library is prepared. In some embodiments, a double-stranded DNA library, a single-stranded DNA library or an RNA library is prepared. A method of preparing a dsDNA library can comprise ligating an adapter sequence onto one or both ends of a dsDNA fragment. In some cases, the adapter sequence comprises a primer docking sequence. In some cases, the method further comprises hybridizing a primer to the primer docking sequence and initiating amplification or sequencing of the nucleic acid attached to the adapter. In some embodiments, the primer or the primer docking sequence comprises at least a portion of an adapter sequence that couples to a next-generation sequencing platform. In some embodiments, a method can further comprise extension of a hybridized primer to create a duplex, wherein a duplex comprises an original ssDNA fragment and an extended primer strand. In some embodiments, an extended primer strand can be separated from an original ssDNA fragment. In some embodiments, an extended primer strand can be collected, wherein an extended primer strand is a member of an ssDNA library.

In some cases, the library is prepared in an unbiased manner. For example, in some cases, the library is prepared without using a primer that specifically hybridizes to a microbial nucleic acid. For example, in some embodiments, the only amplification performed on the sample involves the use of a primer specific for a sequence of one or more adapters attached to nucleic acids within the sample. In some cases, whole genome amplification is used to prepare the library prior to attachment of the adapters. In some cases, whole genome amplification is not used to prepare the library. In some cases, one or more primers that specifically hybridize to a microbial nucleic acid (e.g., pathogen, viral, fungal, bacterial or parasite nucleic acid) are used to amplify the sample.

In some cases, multiple DNA libraries from different samples (e.g., samples from different patients or subjects) are combined and then subjected to a next generation sequencing assay. In some cases, the libraries are indexed prior to combining in order to track which library corresponds to which sample. Indexing can involve the inclusion of a specific code or bar code in an adapter, e.g., an adapter that is attached to the nucleic acids are to be analyzed. In some cases, the samples comprise a negative control sample or a positive control sample, or both a negative control sample and a positive control sample.

In some cases, multiple DNA libraries from different samples (e.g., samples from different patients or subjects) are combined and then subjected to a next generation sequencing assay. In some cases, the samples comprise a negative control sample or a positive control sample.

In some embodiments, a length of a nucleic acid can vary. In some embodiments, a nucleic acid or nucleic acid fragment (e.g., dsDNA fragment, RNA, or randomly sized cDNA) can be less than 1000 bp, less than 800 bp, less than 700 bp, less than 600 bp, less than 500 bp, less than 400 bp, less than 300 bp, less than 200 bp, or less than 100 bp. In some embodiments, a DNA fragment can be about 40 to about 100 bp, about 50 to about 125 bp, about 100 to about 200 bp, about 150 to about 400 bp, about 300 to about 500 bp, about 100 to about 500 bp, about 400 to about 700 bp, about 500 to about 800 bp, about 700 to about 900 bp, about 800 to about 1000 bp, or about 100 to about 1000 bp. In some embodiments, a nucleic acid or nucleic acid fragment (e.g., dsDNA fragment, RNA, or randomly sized cDNA) can be within a range from about 20 to about 200 bp, such as within a range from about 40 to about 100 bp.

In some embodiments, an end of a dsDNA fragment can be polished (e.g., blunt-ended)) or be subject to end-repair to create a blunt end. In some embodiments, an end of a DNA fragment can be polished by treatment with a polymerase. In some embodiments, a polishing can involve removal of a 3′ overhang, a fill-in of a 5′ overhang, or a combination thereof. In some embodiments, a polymerase can be a proof-reading polymerase (e.g., comprising 3′ to 5′ exonuclease activity). In some embodiments, a proofreading polymerase can be, e.g., a T4 DNA polymerase, Pol 1 Klenow fragment, or Pfu polymerase. In some embodiments, a polishing can comprise removal of damaged nucleotides (e.g., abasic sites), using any means known in the art.

In some embodiments, a ligation of an adapter to a 3′ end of a nucleic acid fragment can comprise formation of a bond between a 3′ OH group of the fragment and a 5′ phosphate of the adapter. Therefore, removal of 5′ phosphates from nucleic acid fragments can minimize aberrant ligation of two library members. Accordingly, in some embodiments, 5′ phosphates are removed from nucleic acid fragments. In some embodiments, 5′ phosphates are removed from at least 50%, 55%, 60%, 65%, 70%, 75%, 80%, 85%, 90%, 95%, or greater than 95% of nucleic acid fragments in a sample. In some embodiments, substantially all phosphate groups are removed from nucleic acid fragments. In some embodiments, substantially all phosphates are removed from at least 50%, 55%, 60%, 65%, 70%, 75%, 80%, 85%, 90%, 95%, or greater than 95% of nucleic acid fragments in a sample. Removal of phosphate groups from a nucleic acid sample can be by any means known in the art. Removal of phosphate groups can comprise treating the sample with heat-labile phosphatase. In some embodiments, phosphate groups are not removed from the nucleic acid sample. In some embodiments, ligation of an adapter to the 5′ end of the nucleic acid fragment is performed.

Exemplary Sample Processing and Analysis

What follows is an example of methods provided by this disclosure. In some cases, plasma is spiked with a known concentration of synthetic normalization molecule controls. In some cases, the plasma is then subjected to cell-free NA (cfNA) extraction (e.g., extraction of cell-free DNA). The extracted cfNA can be processed by end-repair and ligated to adapters containing specific indexes to end-repaired cfDNA. The products of the ligation can be purified by beads. In some embodiments, the cfDNA ligated to adapters can be amplified with P5 and P7 primers, and the amplified, adapted cfDNA is purified.

Purified cfDNA attached to adapters derived from a plasma sample can be incorporated into a DNA sequencing library. Sequencing libraries from several plasma samples can be pooled with control samples, purified, and, in some embodiments, sequenced on Illumina sequencers using a 75-cycle single-end, dual index sequencing kit. Primary sequencing output can be demultiplexed followed by quality trimming of the reads. In some embodiments, the reads that pass quality filters are aligned against human and synthetic references and then excluded from the analysis, or otherwise set aside. Reads potentially representing human satellite DNA can also be filtered, e.g., via a k-mer-based method; then the remaining reads can be aligned with a microorganism reference database, (e.g., a database with 20,963 assemblies of high-quality genomic references). In some embodiments, reads with alignments that exhibit both high percent identity and/or high query coverage can be retained, except, e.g., for reads that are aligned with any mitochondrial or plasmid reference sequences. PCR duplicates can removed based on their alignments. Relative abundances can be assigned to each taxon in a sample based on the sequencing reads and their alignments.

For each combination of read and taxon, a read sequence probability can be defined that accounts for the divergence between the microorganism present in the sample and the reference assemblies in the database. A mixture model can be used to assign a likelihood to the complete collection of sequencing reads that included the read sequence probabilities and the (unobserved) abundances of each taxon in the sample. In some cases, an expectation-maximization algorithm is applied to compute the maximum likelihood estimate of each taxon abundance. From these abundances, the number of reads arising from each taxon can be aggregated up the taxonomic tree. The estimated taxa abundances from the no template control (NTC) samples within the batch can be combined to parameterize a model of read abundance arising from the environment with variations driven by counting noise. Statistical significance values can then be computed for each estimate of taxon abundance in each patient sample. In some embodiments, taxa that exhibit a high significance level, and are one of the 1449 taxa within the reportable range, comprise the candidate calls. Final calls can be made after additional filtering is applied, which accounts for read location uniformity as well as cross-reactivity risk originating from higher abundance calls. The microorganism calls that pass these filters are reported along with abundances in MPM, as estimated using the ratio between the unique reads for the taxon and the number of observed unique reads of normalization molecules.

The amount of mcfDNA plasma concentration in each sample can then be quantified by using the measured relative abundance of the synthetic molecules initially spiked in the plasma.

In some cases, testing with plasma mcfDNA-seq is performed on available samples collected between seven days before and four days after each BSI episode, and two negative control samples are added for each BSI episode. In some cases, the samples are collected at least three days prior to a bloodstream infection of invasive fungal infection. The laboratory can be blinded to expected results until sequencing is completed and reported.

Analysis

Disclosed herein in some embodiments, are methods of analyzing nucleic acids. Such analytical methods include sequencing the nucleic acids as well as bioinformatic analysis of the sequencing results (e.g., sequence reads).

In some embodiments, a sequencing is performed using a next generation sequencing assay. As used herein, the term “next generation” generally refers to any high-throughput sequencing approach including, but not limited to one or more of the following: massively-parallel signature sequencing, pyrosequencing (e.g., using a Roche 454 Genome Analyzer™ sequencing device), Illumina™ (Solexa™) sequencing (e.g., using an Illumina NextSeq™ 500), sequencing by synthesis (Illumina™), ion semiconductor sequencing (Ion torrent™), sequencing by ligation (e.g., SOLiD™ sequencing), single molecule real-time (SMRT) sequencing (e.g., Pacific Bioscience™), polony sequencing, DNA nanoball sequencing (Complete Genomics™), heliscope single molecule sequencing (Helicos Biosciences™), and nanopore sequencing (e.g., Oxford Nanopore™). In some embodiments, a sequencing assay can comprise nanopore sequencing. In some embodiments, a sequencing assay can include some form of Sanger sequencing. In some embodiments, a sequencing can involve shotgun sequencing; in some embodiments, a sequencing can include bridge amplification PCR. In some embodiments, a sequencing can be broad spectrum. In some embodiments, a sequencing can be targeted.

In some embodiments, a sequencing assay can comprise a Gilbert's sequencing method. In some embodiments, a Gilbert's sequencing method can comprise chemically modifying nucleic acids (e.g., DNA) and then cleaving them at specific bases. In some embodiments, a sequencing assay can comprise dideoxynucleotide chain termination or Sanger-sequencing.

In some embodiments, a sequencing-by-synthesis approach can be used in the methods provided herein. In some embodiments, fluorescently-labeled reversible-terminator nucleotides are introduced to clonally-amplified DNA templates immobilized on the surface of a glass flowcell. During each sequencing cycle, a single labeled deoxynucleoside triphosphate (dNTP) may be added to the nucleic acid chain. The labeled terminator nucleotide may be imaged when added in order to identify the base and may then be enzymatically cleaved to allow incorporation of the next nucleotide. Since all four reversible terminator-bound dNTPs (A, C, T, G) are generally present as single, separate molecules, natural competition may minimize incorporation bias.

In some embodiments, a method called Single-molecule real-time (SMRT) is used. In such approach, nucleic acids (e.g., DNA) are synthesized in zero-mode wave-guides (ZMWs), which are small well-like containers with capturing tools located at the bottom of the well. The sequencing is performed with use of unmodified polymerase (attached to the ZMW bottom) and fluorescently labelled nucleotides flowing freely in the solution. The fluorescent label is detached from the nucleotide upon its incorporation into the DNA strand, leaving an unmodified DNA strand. A detector such as a camera may then be used to detect the light emissions; and the data may be analyzed bioinformatically to obtain sequence information.

In some embodiments, a sequencing by ligation approach is used to sequence the nucleic acids in a sample. One example is the next generation sequencing method of SOLiD (Sequencing by Oligonucleotide Ligation and Detection) sequencing (Life Technologies). This next generation technology may generate hundreds of millions to billions of small sequence reads at one time. The sequencing method may comprise preparing a library of DNA fragments from the sample to be sequenced. In some embodiments, the library is used to prepare clonal bead populations in which only one species of fragment is present on the surface of each bead (e.g., magnetic bead). The fragments attached to the magnetic beads may have a universal P1 adapter sequence attached so that the starting sequence of every fragment is both known and identical. In some embodiments, the method may further involve PCR or emulsion PCR. For example, the emulsion PCR may involve the use of microreactors containing reagents for PCR. The resulting PCR products attached to the beads may then be covalently bound to a glass slide. A sequencing assay such as a SOLiD sequencing assay or other sequencing by ligation assay may include a step involving the use of primers. Primers may hybridize to the P1 adapter sequence or other sequence within the library template. The method may further involve introducing four fluorescently labelled di-base probes that compete for ligation to the sequencing primer. Specificity of the di-base probe may be achieved by interrogating every first and second base in each ligation reaction. Multiple cycles of ligation, detection and cleavage may be performed with the number of cycles determining the eventual read length. In some embodiments, following a series of ligation cycles, the extension product can be removed and the template can be reset with a primer complementary to the n−1 position for a second round of ligation cycles. Multiple rounds (e.g., 5 rounds) of primer reset may be completed for each sequence tag. Through the primer reset process, each base may be interrogated in two independent ligation reactions by two different primers. For example, a base at read position 5 can be assayed by primer number 2 in ligation cycle 2 and by primer number 3 in ligation cycle 1.

In some embodiments, a detection or quantification analysis of oligonucleotides can be accomplished by sequencing. In some embodiments, entire synthesized oligonucleotides can be detected via full sequencing of all oligonucleotides by e.g., Illumina HiSeq 2500™, including the sequencing methods described herein.

In some embodiments, a sequencing can be accomplished through classic Sanger sequencing methods which are well known in the art. Sequencing can also be accomplished using high-throughput systems some of which allow detection of a sequenced nucleotide immediately after or upon its incorporation into a growing strand, e.g., detection of sequence in real time or substantially real time. In some embodiments, high throughput sequencing generates at least 1,000, at least 5,000, at least 10,000, at least 20,000, at least 30,000, at least 40,000, at least 50,000, at least 100,000, or at least 500,000 sequence reads per hour. In some embodiments, each read is at least 50, at least 60, at least 70, at least 80, at least 90, at least 100, at least 120, or at least 150 bases per read. In some embodiments, each read is up to 2000, up to 1000, up to 900, up to 800, up to 700, up to 600, up to 500, up to 400, up to 300, up to 200, or up to 100 bases per read. Long read sequencing can include sequencing that provides a contiguous sequence read of longer than 500 bases, longer than 800 bases, longer than 1000 bases, longer than 1500 bases, longer than 2000 bases, longer than 3000 bases, or longer than 4500 bases per read.

In some embodiments, a high-throughput sequencing can involve the use of technology available by Illumina's Genome Analyzer IIX™, MiSeq personal sequencer™, or HiSeq™ systems, such as those using HiSeq 2500 ™, HiSeq 1500 ™, HiSeq 2000 ™, or HiSeq 1000 ™. These machines use reversible terminator-based sequencing by synthesis chemistry. These machines can sequence 200 billion or more reads in eight days. Smaller systems may be utilized for runs within 3, 2, or 1 days or less time. Short synthesis cycles may be used to minimize the time it takes to obtain sequencing results.

In some embodiments, a high-throughput sequencing involves the use of technology available by ABI Solid System. This genetic analysis platform can enable massively parallel sequencing of clonally-amplified DNA fragments linked to beads. The sequencing methodology is based on sequential ligation with dye-labeled oligonucleotides.

In some embodiments, a next-generation sequencing can comprise ion semiconductor sequencing (e.g., using technology from Life Technologies™ (Ion Torrent™)). Ion semiconductor sequencing can take advantage of the fact that when a nucleotide is incorporated into a strand of DNA, an ion can be released.

To perform ion semiconductor sequencing, a high density array of micromachined wells can be formed. Each well can hold a single DNA template. Beneath the well can be an ion sensitive layer, and beneath the ion sensitive layer can be an ion sensor. When a nucleotide is added to a DNA, an H+ ion can be released, which can be measured as a change in pH. The H+ ion can be converted to voltage and recorded by the semiconductor sensor. An array chip can be sequentially flooded with one nucleotide after another. In some embodiments, no scanning, light, or cameras are required. In some embodiments, an IONPROTON™ Sequencer is used to sequence nucleic acid. In some embodiments, an IONPGM™ Sequencer is used. The Ion Torrent Personal Genome Machine™ (PGM) can sequence 10 million reads in two hours.

In some embodiments, a high-throughput sequencing involves the use of technology available by Helicos BioSciences Corporation™ (Cambridge, Massachusetts) such as the Single Molecule Sequencing by Synthesis (SMSS) method. SMSS can allow for sequencing the entire human genome in up to 24 hours. In some embodiments, SMSS may not require a pre amplification step prior to hybridization. In some embodiments, SMSS may not require any amplification. In some embodiments, methods of using SMSS are described in part in US Publication Application Nos. 20060024711 which is herein incorporated by reference.

In some embodiments, a high-throughput sequencing involves the use of technology available by 454 Lifesciences, Inc.™ (Branford, Connecticut) such as the Pico Titer Plate™ device which includes a fiber optic plate that transmits chemiluminescent signal generated by the sequencing reaction to be recorded by a charge-coupled device (CCD) camera in the instrument. This use of fiber optics can allow for the detection of a minimum of 20 million base pairs in 4.5 hours. In some embodiments, methods for using bead amplification followed by fiber optics detection are described in US Publication Application Nos. 20020012930; 20030058629; 20030100102; 20030148344; 20040248161; 20050079510, 20050124022; and 20060078909, each of which are herein incorporated by reference.

In some embodiments, high-throughput sequencing is performed using Clonal Single Molecule Array (Solexa, Inc.™) or sequencing-by-synthesis (SBS) utilizing reversible terminator chemistry.

In some embodiments, the next generation sequencing is nanopore sequencing. A nanopore can be a small hole, e.g., on the order of about one nanometer in diameter. Immersion of a nanopore in a conducting fluid and application of a potential across it can result in a slight electrical current due to conduction of ions through the nanopore. The amount of current which flows can be sensitive to the size of the nanopore. As a DNA molecule passes through a nanopore, each nucleotide on the DNA molecule can obstruct the nanopore to a different degree. Thus, the change in the current passing through the nanopore as the DNA molecule passes through the nanopore can represent a reading of the DNA sequence. The nanopore sequencing technology can be from Oxford Nanopore Technologies™; e.g., a GridION™ system. A single nanopore can be inserted in a polymer membrane across the top of a microwell. Each microwell can have an electrode for individual sensing. The microwells can be fabricated into an array chip, with 100,000 or more microwells (e.g., more than 200,000, 300,000, 400,000, 500,000, 600,000, 700,000, 800,000, 900,000, or 1,000,000) per chip. An instrument (or node) can be used to analyze the chip. Data can be analyzed in real-time. One or more instruments can be operated at a time. The nanopore can be a protein nanopore, e.g., the protein alpha-hemolysin, a heptameric protein pore. The nanopore can be a solid-state nanopore made, e.g., a nanometer sized hole formed in a synthetic membrane (e.g., SiNx, or SiO2). The nanopore can be a hybrid pore (e.g., an integration of a protein pore into a solid-state membrane). The nanopore can be a nanopore with an integrated sensors (e.g., tunneling electrode detectors, capacitive detectors, or graphene based nano-gap or edge state detectors (see e.g., Garaj et al. (2010) Nature vol. 67, doi: 10.1038/nature09379)). A nanopore can be functionalized for analyzing a specific type of molecule (e.g., DNA, RNA, or protein). Nanopore sequencing can comprise “strand sequencing” in which intact DNA polymers can be passed through a protein nanopore with sequencing in real time as the DNA translocates the pore. An enzyme can separate strands of a double stranded DNA and feed a strand through a nanopore. The DNA can have a hairpin at one end, and the system can read both strands. In some embodiments, nanopore sequencing is “exonuclease sequencing” in which individual nucleotides can be cleaved from a DNA strand by a processive exonuclease, and the nucleotides can be passed through a protein nanopore. The nucleotides can transiently bind to a molecule in the pore (e.g., cyclodextran). A characteristic disruption in current can be used to identify bases. Methods of using these technologies are described in part in Soni G V and Meller A. (2007) Clin Chem 53: 1996-2001, which are herein incorporated by reference.

In some embodiments, a nanopore sequencing technology from GENIA™ can be used. An engineered protein pore can be embedded in a lipid bilayer membrane. “Active Control” technology can be used to enable efficient nanopore-membrane assembly and control of DNA movement through the channel. In some embodiments, the nanopore sequencing technology is from NABsys™. Genomic DNA can be fragmented into strands of average length of about 100 kb. The 100 kb fragments can be made single stranded and subsequently hybridized with a 6-mer probe. The genomic fragments with probes can be driven through a nanopore, which can create a current-versus-time tracing. The current tracing can provide the positions of the probes on each genomic fragment. The genomic fragments can be lined up to create a probe map for the genome. The process can be done in parallel for a library of probes. A genome-length probe map for each probe can be generated. Errors can be fixed with a process termed “moving window Sequencing By Hybridization (mwSBH).” In some embodiments, the nanopore sequencing technology is from IBM™ or Roche™. An electron beam can be used to make a nanopore sized opening in a microchip. An electrical field can be used to pull or thread DNA through the nanopore. A DNA transistor device in the nanopore can comprise alternating nanometer sized layers of metal and dielectric. Discrete charges in the DNA backbone can get trapped by electrical fields inside the DNA nanopore. Turning off and on gate voltages can allow the DNA sequence to be read.

The next generation sequencing can comprise DNA nanoball sequencing (as performed, e.g., by Complete Genomics™; see e.g., Drmanac et al. (2010) Science 327: 78-81, which is incorporated herein by reference). DNA can be isolated, fragmented, and size selected. For example, DNA can be fragmented (e.g., by sonication) to a mean length of about 500 bp. Adapters (Ad1) can be attached to the ends of the fragments.

The adapters can be used to hybridize to anchors for sequencing reactions. DNA with adapters bound to each end can be PCR amplified. The adapter sequences can be modified so that complementary single strand ends bind to each other forming circular DNA. The DNA can be methylated to protect it from cleavage by a type IIS restriction enzyme used in a subsequent step. An adapter (e.g., the right adapter) can have a restriction recognition site, and the restriction recognition site can remain non-methylated. The non-methylated restriction recognition site in the adapter can be recognized by a restriction enzyme (e.g., Acul), and the DNA can be cleaved by Acul 13 bp to the right of the right adapter to form linear double stranded DNA. A second round of right and left adapters (Ad2) can be ligated onto either end of the linear DNA, and all DNA with both adapters bound can be PCR amplified (e.g., by PCR). Ad2 sequences can be modified to allow them to bind each other and form circular DNA. The DNA can be methylated, but a restriction enzyme recognition site can remain non-methylated on the left Ad1 adapter. A restriction enzyme (e.g., Acul) can be applied, and the DNA can be cleaved 13 bp to the left of the Ad1 to form a linear DNA fragment. A third round of right and left adapter (Ad3) can be ligated to the right and left flank of the linear DNA, and the resulting fragment can be PCR amplified. The adapters can be modified so that they can bind to each other and form circular DNA. A type III restriction enzyme (e.g., EcoP15) can be added; EcoP15 can cleave the DNA 26 bp to the left of Ad3 and 26 bp to the right of Ad2. This cleavage can remove a large segment of DNA and linearize the DNA once again. A fourth round of right and left adapters (Ad4) can be ligated to the DNA, the DNA can be amplified (e.g., by PCR), and modified so that they bind each other and form the completed circular DNA template.

Rolling circle replication (e.g., using Phi 29 DNA polymerase) can be used to amplify small fragments of DNA. The four adapter sequences can contain palindromic sequences that can hybridize and a single strand can fold onto itself to form a DNA nanoball (DNB™) which can be approximately 200-300 nanometers in diameter on average. A DNA nanoball can be attached (e.g., by adsorption) to a microarray (sequencing flowcell). The flow cell can be a silicon wafer coated with silicon dioxide, titanium and hexamethyldisilazane (HMDS) and a photoresistant material. Sequencing can be performed by unchained sequencing by ligating fluorescent probes to the DNA. The color of the fluorescence of an interrogated position can be visualized by a high resolution camera. The identity of nucleotide sequences between adapter sequences can be determined.

The methods provided herein may include use of a system that contains a nucleic acid sequencer (e.g., DNA sequencer, RNA sequencer) for generating DNA or RNA sequence information. The system may include a computer comprising software that performs bioinformatic analysis on the DNA or RNA sequence information. Bioinformatic analysis can include, without limitation, assembling sequence data, detecting and quantifying genetic variants in a sample, including germline variants and somatic cell variants (e.g., a genetic variation associated with cancer or pre-cancerous condition, a genetic variation associated with infection, or a combination thereof).

Sequencing data may be used to determine genetic sequence information, ploidy states, the identity of one or more genetic variants, as well as a quantitative measures of the variants, including relative and absolute relative measures.

In some embodiments, a sequencing can involve sequencing of a genome. In some embodiments, a genome can be that of a pathogen as disclosed herein. In some embodiments, sequencing of a genome can involve whole genome sequencing or partial genome sequencing. In some embodiments, a sequencing can be unbiased and can involve sequencing all or substantially all (e.g., greater than 70%, 80%, 90%) of the nucleic acids in a sample. In some embodiments, a sequencing of a genome can be selective, e.g., directed to portions of a genome of interest. In some embodiments, sequencing of select genes, or portions of genes may suffice for a desired analysis. In some embodiments, polynucleotides mapping to specific loci in a genome can be isolated for sequencing by, for example, sequence capture or site-specific amplification.

In some embodiments, disclosed herein, is a method comprising a process of analyzing, calculating, quantifying, or a combination thereof. In some embodiments, a method can be used to determine quantities of bacterial and fungal sequence reads. In some embodiments, metrics can be generated to determine quantities of bacterial sequences, fungal sequences or a combination thereof.

In some embodiments, the quantity for each organism identified in a method provided herein is expressed in Molecules Per Microliter of biological fluid (e.g., plasma) (MPM), the number of DNA sequencing reads from the reported organism present per microliter of plasma. In some cases, detection or prediction of infection (or of severity of infection or of hyper-inflammatory response or of mortality from COVID-19) occurs when the MPM is greater than a threshold value. In some cases, such threshold value of MPM is 10, 15, 20, 30, 50, 100, 200, 300, 400, 500, 600, 700, 800, 900, 1000, 2000, 3000, 3500, 4000, 4500, 5000, 7000, 10000, 20000, 30000, or 40000. In some cases, the threshold value is 100 MPM. In some cases, the threshold value is 100 MPM. In some cases, total MPM (e.g., total MPM from respiratory pathogens) above 100 MPM is indicative of a secondary infection. In some cases, total MPM above 100 MPM is indicative of a hyperinflammatory response. In some cases, the threshold value is 400 MPM. In some cases, total MPM (e.g., total MPM from respiratory pathogens) above 400 MPM is indicative of a secondary infection. In some cases, total MPM above 400 MPM is indicative of a hyperinflammatory response. In some cases, the threshold value is 3000 MPM. In some cases, total MPM (e.g., total MPM from respiratory pathogens) above 3000 MPM is indicative of a secondary infection. In some cases, total MPM above 3000 MPM is indicative of a hyperinflammatory response. In some cases, the threshold value is 4000 MPM. In some cases, total MPM (e.g., total MPM from respiratory pathogens) above 4000 MPM is indicative of a secondary infection. In some cases, total MPM above 4000 MPM is indicative of a hyperinflammatory response. In some cases, such threshold value of MPM is at least (or greater than) 10, 15, 20, 30, 50, 100, 200, 300, 400, 500, 600, 700, 800, 900, 1000, 2000, 3000, 3500, 4000, 4500, 5000, 7000, 10000, 20000, 30000, or 40000. In some cases, the MPM threshold is determined for a particular organism. In some cases, the MPM threshold is a value that is an aggregate amount of mcfNA (e.g., mcfDNA) from more than one single organism (e.g., aggregate amount of mcfNA from bacteria, from respiratory pathogens, from respiratory bacteria, from bacteria and fungi, or from a specific set of pathogens). In some embodiments, the respiratory pathogen is at least one respiratory pathogen listed in Table 2, in any combination. In some embodiments, the respiratory pathogen is a Streptococcus, Pseudomonas, or Klebsiella bacterium. In some embodiments, the respiratory pathogen is from any genus listed in Table 2. In some cases, the respiratory pathogen is from the genus Actinomyces, Aspergillus, Bacteroides, Citrobacter, Cytomegalovirus, Enterobacter, Escherichia, Enterococcus, Streptococcus, Pseudomonas, Klebsiella, and/or Haemophilus, In some cases, the respiratory pathogen is S. aureus, P. aeruginosa and/or K. pneumoniae, in any combination. In some cases, the MPM threshold for any of the preceding infections is “about” (as defined herein) any of the preceding values.

In some cases, the MPM threshold represents the MPM for an uninfected or healthy control. In some cases, the MPM threshold refers to a threshold indicative of disease severity or risk of mortality (e.g., greater than 1000, 4000, 5000, 7000, or 10000) may indicate a high risk of non-survival from Covid-19.

Sequencing Systems

This disclosure also provides sequencing systems for nucleic acid or DNA sequencing. In some embodiments, the nucleic acid sequencing system is for detecting secondary infection in a subject with a first infection. In some embodiments, the system comprises a next-generation sequencing device comprising a flow cell and a computer processor that outputs data comprising sequence reads collected from measurements conducted in the flow cell. In some embodiments, the system comprises or further comprises a computing device that comprises quantitation of total microbial cell-free nucleic acids (mcfNA) logic that (i) detects mcfNA from at least two different microbes by aligning the sequence reads to microbial reference sequence reads; (ii) calculates total mcfNA as a function of molecules per microliter of plasma, wherein the total mcfNA is an aggregate value of mcfNA from the at least two different microbes; and (iii) comprises an event generator to generate an event indicative a secondary infection when the total mcfNA exceeds a threshold value. In some cases, the genomic references include sequences from pathogens in Table 2.

In some cases, the threshold value is at least 50 MPM, 70 MPM, 100 MPM, 200 MPM, 500 MPM, 1000 MPM, 2000 MPM, 3000 MPM, 4000 MPM, 5000 MPM, 10000 MPM, 50000 MPM, or 100000 MPM. In some cases, the threshold value that is “about” any of the preceding MPM values. In some cases, the threshold value is the value associated with MPM for microbial cell-free nucleic acids (e.g, mcfDNA) from a healthy or uninfected subject, or subject that has a hypo-inflammatory response.

Treatments

In some embodiments, the non-limiting methods provided herein can comprise administering a treatment to a subject. In some cases, the treatment treats a disease or disorder, such as by reducing symptoms or signs of the disease or disorder. In some cases, the disease or disorder is an infection (e.g., bacterial infection, fungal infection, respiratory infection, pneumonia, bacterial pneumonia, viral pneumonia). In some cases, the disease or disorder is inflammation. In some cases, the treating occurs prior to onset of an infection or inflammation and, in some embodiments, prior to onset of one or more symptoms of infection (e.g., fever, elevated heart rate, low blood pressure, hyperventilation). In some embodiments, the treatment is administered to a subject when the subject is blood culture negative for the organism that is the target of the treatment. In some embodiments, the infection is detected or predicted by a method provided herein when the subject is blood culture negative, but the treatment is administered when the subject is blood culture positive. In some embodiments, the infection is detected or predicted by a method provided herein when the subject is blood culture negative, and the treatment is administered when the subject is blood culture negative. In some embodiments, the infection is detected or predicted by a method provided herein when the subject is blood culture positive, and the treatment is administered when the subject is blood culture positive. In some cases, the treatment is provided when the subject has not had a blood culture, or when the blood culture is non-conclusive. In some embodiments, the treatment is a preemptive treatment that prevents an asymptomatic infection from progressing into a symptomatic infection. In some embodiments, the treatment is a prophylactic treatment that prevents the onset of infection. In some embodiments, the treatment treats or reduces symptoms of an infection.

Various non-limiting treatments provided herein can be administered to the subject. In some embodiments, the treatment is a broad-spectrum antimicrobial drug or an antimicrobial drug that targets a specific microbe or a specific class of microbes. In some embodiments, the treatment targets bacteria and/or fungi, particularly any of the microbial organisms identified herein (e.g, in the Examples section of this application). In some embodiments, the subject is treated with a combination of drugs (e.g., a combination of multiple antibiotics, multiple anti-fungal drugs, or both antibiotics and antifungal drugs). In some embodiments, the subject is treated with a combination of broad-spectrum antibiotics, a combination of broad- and narrow-spectrum antibiotics, a combination of narrow-spectrum antibiotics, a combination of broad-spectrum antifungals, a combination of broad and narrow-spectrum antifungals, or a combination of narrow-spectrum antifungals. In some embodiments, the subject is treated with a broad-spectrum antibiotic, a narrow-spectrum antibiotic, a broad-spectrum antifungal, a narrow-spectrum antifungal, or any combination thereof.

In some embodiments, the treatment is an antimicrobial. In some embodiments, the antimicrobial comprises a beta-lactam, an aminoglycoside, a quinolone, an oxazolidinone, a sulfonamide, a macrolide, a tetracycline, an ansamycin, a streptogramin, a lipopeptide, used singly, or in any combination thereof as used herein and/or as recommended by a clinician. In some embodiments, the treatment is a broad-spectrum treatment. In some embodiments, the broad-spectrum treatment is a broad-spectrum antibiotic, a broad-spectrum anti-bacterial drug, a broad-spectrum antifungal, or any combination thereof. As used herein, the term “broad spectrum antibiotic” generally refers to a drug that acts on both gram negative and gram-positive bacteria, that acts on multiple types of gram-negative bacteria, and/or that acts on multiple types of gram-positive bacteria. In some embodiments, the broad-spectrum treatment acts on multiple types of fungal infections. In some embodiments, the drug is a beta-lactam penicillin such as flucloxacillin, ampicillin (or amoxicillin). In some embodiments, the broad-spectrum drug is a beta-lactam such as cephalosporin antibiotic (e.g., ceftriaxone, cefepime). The cephalosporin drug can be, in some embodiments, a first, second, third or fourth generation cephalosporin drug. In some embodiments, the broad-spectrum antibiotic is a quinolone drug (e.g., levofloxacin), a carbopenem-type antibiotic (e.g., meropenem), or a metronidazole.

In some cases, the treatment is an antibiotic. In some embodiments, the treatment is a glycopeptidic antibiotic active against gram-positive bacteria. For example, in some embodiments, the treatment is vancomycin. In some embodiments, the treatment comprises one or more antibiotics listed in Table 5.

In some embodiments, the treatment is an anti-fungal drug. In some embodiments, the treatment is a broad-spectrum antifungal drug. In some embodiments, the antifungal drug is, for example, a cefepime, a clotrimazole, an econazole, a miconazole, a terbinafine, a fluconazole, a ketoconazole, a nystatin, an amphotericin B, or any other known antifungal drugs and/or a combination thereof.

In some embodiments, the treatment comprises various narrow-spectrum drugs, for example, a flucytosine. In some embodiments, the narrow-spectrum drug is an oxazolidinone, for example, a linezolid, a posizolid, a radezolid, a penicillin VK, or any combination thereof.

In some embodiments, the antimicrobial drug is a pill, a gel, a tablet, a coated tablet, or any combination thereof and can be administered to the subject orally. In some embodiments, the treatment using an anti-fungal can be administered to the subject topically. In some embodiments, a treatment can be administered in the form of a capsule, a tablet, a liquid, an injectable, a pessary or any combination thereof. In some embodiments, the antimicrobial drug is formulated as an infusion, and can be administered to the subject intravenously via a needle or catheter.

In some cases, the treatment is an anti-inflammatory drug. For example, in some cases, the treatment is a non-steroidal anti-inflammatory drug (NSAID). In some cases, the anti-inflammatory drug is a steroid. In some cases, the drug is a corticosteroid. In some cases, the drug is dexamethasone. In some cases, the drug is prednisone.

In some cases, the treatment is a treatment for COVID-19. In some cases, the treatment is remdesivir. In some cases, the drug is a monoclonal antibody. In some cases, a method provided herein may indicate that the subject has a risk of severe COVID-19 or a risk of not surviving COVID-19, and the subject may be administered a drug to treat or prevent the severe COVID-19, such as remdesivir or a mono-clonal antibody.

### EXAMPLES

The present invention is described in further detain in the following examples which are not in any way intended to limit the scope of the invention as claimed. The attached Figures are meant to be considered as integral parts of the specification and description of the invention. All references cited are herein specifically incorporated by reference for all that is described therein. The following examples are offered to illustrate, but not to limit the claimed invention.

In the experimental disclosure which follows, the following abbreviations apply: eq (equivalents); M (Molar); μM (micromolar); N (Normal); mol (moles); mmol (millimoles); pmol (micromoles); nmol (nanomoles); g (grams); mg (milligrams); kg (kilograms); μg (micrograms); L (liters); ml (milliliters); μl (microliters); cm (centimeters); mm (millimeters); μm (micrometers); nm (nanometers); ° C. (degrees Centigrade); h (hours); min (minutes); sec (seconds); msec (milliseconds).

### Example 1

This example illustrates plasma mcfDNA metagenomic sequencing. As previously described, plasma mcfDNA metagenomic sequencing can be performed according to Blauwkamp 2019.

Briefly, plasma is spiked with a known concentration of synthetic normalization molecule controls, followed by cell-free DNA extraction. The extracted cfDNA is processed by end-repair and ligated to adapters containing specific indexes to end-repaired cfDNA. The products of the ligation are purified by beads. The cfDNA attached to adapters is amplified with P5 and P7 primers, and the amplified cfDNA is purified.

Purified cfDNA derived from a plasma sample is incorporated into a DNA sequencing library. Sequencing libraries from several plasma samples can be pooled with control samples, purified, and sequenced on Illumina sequencers using a 75-cycle single-end, dual index sequencing kit. Primary sequencing output is demultiplexed, then the reads are quality trimmed, and reads that pass quality filters are aligned against human and synthetic references and set aside. Reads potentially representing human satellite DNA are also filtered via a k-mer-based method; then the remaining reads are aligned with a microorganism reference database, which consists of 20,963 assemblies of high-quality genomic references. Reads with alignments that exhibit both high percent identity and high query coverage are retained, except for reads that are aligned with any mitochondrial or plasmid reference sequences. PCR duplicates are removed based on their alignments. Relative abundances are assigned to each taxon in a sample based on the sequencing reads and their alignments.

For each combination of read and taxon, a read sequence probability is defined that accounts for the divergence between the microorganism present in the sample and the reference assemblies in the database. A mixture model is used to assign a likelihood to the complete collection of sequencing reads that included the read sequence probabilities and the (unobserved) abundances of each taxon in the sample. An expectation-maximization algorithm can be applied to compute the maximum likelihood estimate of each taxon abundance. From these abundances, the number of reads arising from each taxon is aggregated up the taxonomic tree. The estimated taxa abundances from the no template control (NTC) samples within the batch are combined to parameterize a model of read abundance arising from the environment with variations driven by counting noise. Statistical significance values are then computed for each estimate of taxon abundance in each patient sample. Taxa that exhibit a high significance level, and that are one of the 1449 taxa within the reportable range, comprise our candidate calls. Final calls are made after additional filtering is applied, which accounts for read location uniformity as well as cross-reactivity risk originating from higher abundance calls. The microorganism calls that pass these filters are reported along with abundances in MPM, as estimated using the ratio between the unique reads for the taxon and the number of observed unique reads of normalization molecules.

The amount of mcfDNA plasma concentration in each sample is quantified by using the measured relative abundance of the synthetic molecules initially spiked in the plasma.

### Example 2

Forty-two hospitalized patients with COVID-19 were prospectively enrolled and compared with a historical cohort of mechanically ventilated patients with culture-positive (n=27) vs. culture-negative pneumonia (n=40) or no clinical infection (n=18 controls). From plasma samples, mcfDNA-Seq was used to measure ten host response biomarkers of innate immunity and epithelial/endothelial injury (IL-6, IL-8, IL-10, RAGE, TNFR1, Angiopoietin-2, Procalcitonin, Fractalkine, Pentraxin-3, ST2). Levels of mcfDNA was compared between clinical groups and associations of mcfDNA and biomarker levels were examined with linear regression models.

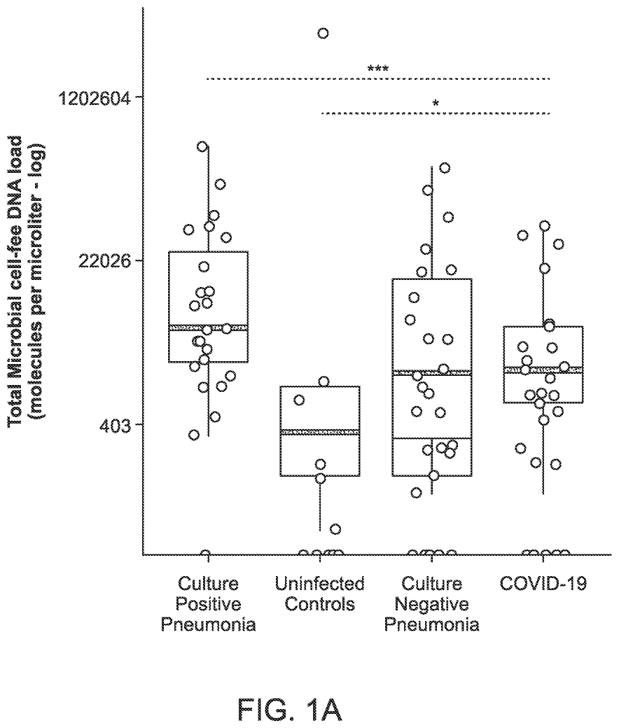

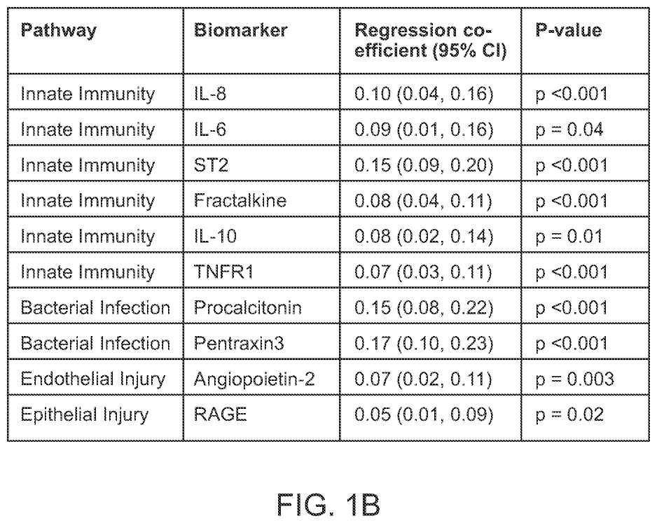

McfDNA-Seq was successful in 33/42 (79%) baseline samples from patients with COVID-19, with nine samples failing QC requirements. McfDNA was detectable in 21/33 (64%) of COVID-19 samples, a proportion significantly lower to culture-positive pneumonia (96%), higher than uninfected controls (33%) and like culture-negative pneumonia (56%) (between-groups Fisher's exact p<0.001). A similar distribution was seen for mcfDNA levels, with mcfDNA load in COVID-19 being similarly distributed to non-COVID culture-negative pneumonia (FIG. 1A). McfDNA was significantly associated with higher levels of host response biomarkers (FIG. 1B), with stronger effect sizes observed for biomarkers of innate immunity (IL-8 and ST2) and bacterial infections (procalcitonin and pentraxin-3).

Plasma metagenomics in patients with COVID-19 revealed mcfDNA load of similar magnitude as in critically ill patients without COVID-19 with clinically suspected infection but negative microbiologic cultures. The significant associations of mcfDNA with host inflammation support the biological relevance of detectable circulating mcfDNA. Our preliminary results warrant further study of secondary infections in hospitalized patients with COVID-19 to define the clinical utility of non-invasive molecular diagnostics for antimicrobial treatment guidance.

### Example 3

Fifteen critically ill patients with COVID-19 (confirmed by nasopharyngeal qPCR for SARS-CoV-2) were enrolled in a prospective ICU cohort study. Plasma samples for conducting mcfDNA-Seq were analyzed according to the methods described in Blauwkamp, 2019, Nature Microbiol, 4:663-74 incorporated by reference herein. Detection of mcfDNA was evaluated in the context of clinical diagnoses and prescribed antimicrobial therapies by the treating physicians and examined for associations with clinical outcomes.

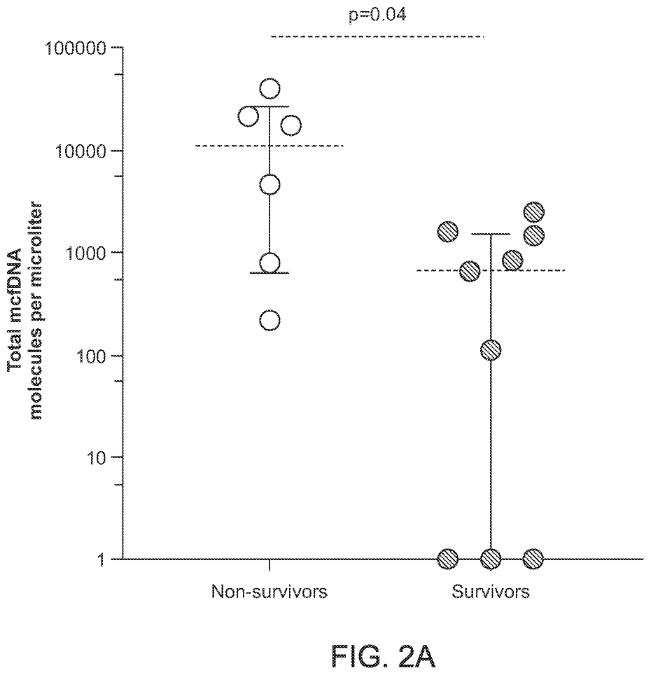

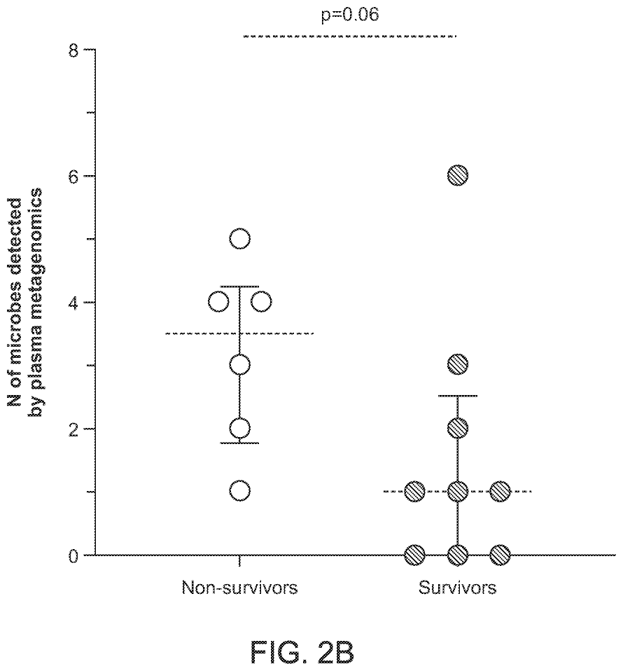

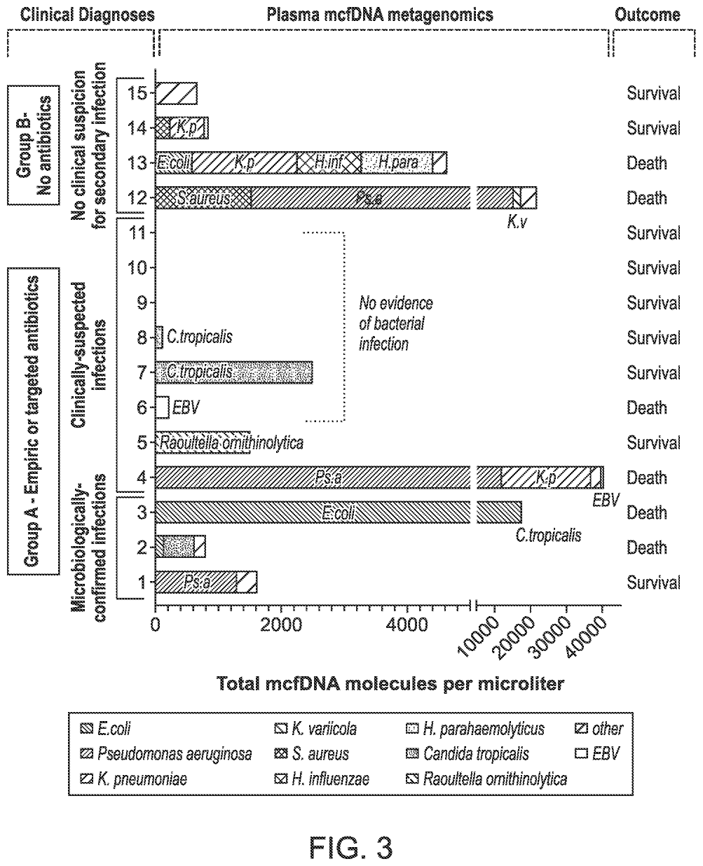

Of fifteen patients analyzed (median age 63, 53% females, 73% mechanically ventilated), six (40%) died within 30 days from enrollment. Samples were obtained at a median (interquartile range-IQR) of ten (4-12) days from COVID-19 symptoms onset, and each sample contained a median of 837 (111-4638) total mcfDNA molecules per microliter (MPMs) and 2 (1-4) identified organisms. Of the total 92,791 MPMs reported across fifteen samples, 90% belonged to typical pathogenic bacteria (e.g., E. coli and K. pneumoniae), with the remainder MPMs aligned to commensal bacteria (5%, e.g., oral Streptococcus species), fungi (4%, Candida species) and DNA viruses (1%). Compared to survivors, non-survivors had higher total mcfDNA (p=0.04), higher pathogenic bacteria MPMs (p=0.02) and a trend for a higher number of identified organisms per sample (p=0.06). (FIG. 2). Secondary pneumonia was clinically suspected or diagnosed by the treating physicians in 11/15 (73%) patients (Group A, FIG. 3), with microbiologic confirmation by positive respiratory cultures in 3/11 subjects (27%); these three patients had high plasma mcfDNA MPMs for common bacterial pathogens, such as E. coli and Ps. aeruginosa. Among the remaining eight patients with clinically suspected infections and empiric antibiotic treatments, high mcfDNA MPMs of probable bacterial pathogens were detected in 2/8 patients (co-infecting Ps. aeruginosa and K. pneumoniae; Raoultella ornithinolytica, respectively). In the additional six patients, no evidence of co-infecting bacterial pathogens was present, whereas in one patient (subject 7, FIG. 2) there was high signal for Candida tropicalis (2,490 MPMs) concerning for undiagnosed invasive Candidiasis. FIG. 2A and FIG. 2B show non-survivors of severe COVID-19 infection had higher microbial cell-free DNA molecules per microliter of plasma by metagenomic sequencing compared to survivors(median [interquartile range]: 11,125 [650-26,436] vs. 661 [1], Wilcoxon test p-value=0.04) and a trend for higher number of identified microbes per sample (3.5 [1.8-4.3] vs. 1.0 [0-2.5], Wilcoxon test p-value=0.06). FIG. 2A shows total mcfDNA molecules per microliter.

FIG. 2B shows N of microbes detected by plasma metagenomics.

Respiratory pathogen MPMs (S. aureus, Ps. aeruginosa and K. pneumoniae) were detected in 3/4 subjects with low suspicion for secondary infection (Group B, FIG. 3). In these patients, no respiratory specimen cultures were obtained, and antibiotics had not been initiated or had been discontinued based on negative blood cultures by the time of research sampling. Two of these individuals experienced sustained vasodilatory shock and died from multiorgan dysfunction attributed to isolated SARS-CoV-2 infection. FIG. 3 shows case-based analysis of 15 critically ill patients with COVID-19 with depicted clinical diagnoses, plasma microbial cell-free DNA metagenomics and survival outcomes. The Y-axis margin indicates two groups of clinical diagnoses: Group A includes eleven patients who received antibiotics for either microbiologically confirmed (n=3) or clinically suspected infections despite negative microbiologic workup (n=8), whereas Group B includes four patients with low clinical suspicion for secondary infection and no antibiotic therapies at time of sampling. The Y-axis ticks denote each patient sample, and the x-height of each stacked bar represents the number of microbial cell-free DNA molecules per plasma microliter (MPMs) by metagenomic sequencing, with different colors for the top ten microbes by ranked abundance. The “other” category (shown in grey) represents the sum of lower abundance taxa of commensal origin. Five out of eleven subjects of Group A (45%, Subjects 1-5) had high MPM signal for probable respiratory pathogens, whereas in the remaining 6/11 subjects there was no evidence of co-infecting bacterial pathogens. Subject 7 was clinically-diagnosed with culture-negative sepsis and treated with prolonged course of empiric broad-spectrum antibiotics while on extracorporeal membrane oxygenation support for refractory hypoxemic respiratory failure from COVID-19; the high mcfDNA signal for C. tropicalis (2,490 MPMs) is concerning for undiagnosed invasive Candidiasis, corroborated by persistent growth of yeast organisms (not further speciated) from clinical bronchoalveolar lavage samples obtained on days 5, 9 and 14 after the research sample acquisition. Two out of four patients of Group B (subjects 12 and 13) who did not survive and had not received empiric antimicrobials were found to have high mcfDNA signal (>4000 total MPMs) of probable respiratory pathogens, indicative of undiagnosed (and untreated) secondary infections.

McfDNA-Seq in patients with COVID-19 indicates a higher incidence of probable secondary infections than previously recognized. The significant association between mcfDNA and 30-day mortality suggests that COVID-19 severity may be influenced by circulating bacterial fragments, either from secondary pneumonias or from possible translocation of colonizing microbiota along the disrupted alveolar/epithelial surface of lungs injured by COVID-19. Kitsios, 2019, Open Forum Infect Dis, 6:S138. Integration of mcfDNA detection with clinical data demonstrates opportunity for antibiotic stewardship in patients with suspected infection. On the other hand, the signal for undiagnosed and untreated secondary infections should serve as a call for vigilance and thorough diagnostic workup in patients with severe COVID-19.

### Example 4

A nested case-control study of mechanically ventilated patients with and without severe pneumonia from an ICU cohort was conducted. Community or hospital-acquired pneumonia were defined per established criteria (Gong, 2005, Crit Care Med, 33:1191-98). Classified patients were defined as culture-positive when pathogenic microbial species were isolated from respiratory specimen or blood cultures vs. culture-negative when no growth in neither culture, or only normal respiratory flora were reported in respiratory cultures. The radiologic severity index (RSI) was quantified on the first available chest radiograph post-intubation and calculated clinical pulmonary infection scores (CPIS) from available data. See Zilberberg, 2010, Clin Infect Dis, 51, S131-35; and Sheshadri, 2019, BMJ Open Respir Res, 6:e000471, herein incorporated by reference in their entirety. Uninfected controls were patients intubated for airway protection or for hypoxemia from decompensated congestive heart failure. Plasma mcfDNA metagenomics was conducted as disclosed in Example 1. Nine host-response biomarkers were measured, and patients were classified in a hyper- vs. hypo-inflammatory sub-phenotype. Metagenomic sequences were quantified as mcfDNA molecules per microliter (MPMs). Clinical variables were compared with biomarker and mcfDNA levels between the three clinical groups (culture-positive pneumonia, culture-negative pneumonia, and uninfected controls) with non-parametric tests and post-hoc adjustments for pairwise comparisons. Associations between biomarkers and mcfDNA concentration (MPMs) were examined with multivariate adjusted linear models following log transformation.

Clinical cohort and sample collection—A convenience sample of consecutive, adult patients intubated and mechanically ventilated was prospectively enrolled. Upon enrollment blood samples were collected for centrifugation, separation of plasma and quantification of host inflammation response biomarkers as well as mcfDNA metagenomic sequencing.

Plasma biomarker measurement—A custom Luminex multi-analyte panel (R&D Systems, Minnesota) was constructed to measure plasma levels of biomarkers with established prognostic utility in pneumonia and Acute Respiratory Distress Syndrome (ARDS), including fractalkine, interleukin (IL)-6, IL-8, pentraxin-3, procalcitonin, receptor for advanced glycation end products (RAGE), suppression of tumorgenicity (ST)-2, and tumor necrosis factor receptor (TNFR)-1.

Hyper- and hypo-inflammation sub-phenotype assignment A 4-variable parsimonious model was used for classification of patients into a hyper- vs. hypo-inflammatory sub-phenotype of host-responses, previously defined by latent class analysis utilizing several clinical and biomarker variables. Drohan, 2020, Host-Response Subphenotypic Classification with A Parsimonious Model Offers Prognostic Information in Patients with Acute Respiratory Failure: A Prospective Cohort Study, doi:10.21203/rs.3.rs-57907/v1. The logit of the probability of hypo-inflammatory sub-phenotype classification was calculated as 0.8739604−8.798345e-05*(angiopoietin-2)−6.049412e-04*(procalcitonin)−4.048723e04*(TNFR-1)+2.883218e-01*(bicarbonate).

### Example 5

To determine an association of mcfDNA and biomarker with inflammation prognosis, twenty-seven culture-positive pneumonia patients, forty culture-negative pneumonia patients, and sixteen uninfected controls were examined. Data of Table 1 are presented as median with interquartile ranges for continuous variables and N with percentage for categorical variables. P-values for comparisons between the three clinical categories were obtained from Kruskal Wallis test for continuous variables and Fisher's exact test for categorical variables. P-values for the comparison between culture-positive vs. -negative pneumonia patients were adjusted for multiple testing with Benjamini-Hochberg correction post-hoc from three group comparisons. P-values for the comparison between patients with pneumonia (both culture-positive and negative) vs. controls were obtained from Wilcoxon test for continuous variables and Fisher's exact test for categorical variables. Among the sixteen uninfected controls, twelve patients were intubated for airway protection without any evidence of respiratory infection, and the remaining four were intubated for cardiogenic pulmonary edema from decompensated congestive heart failure.

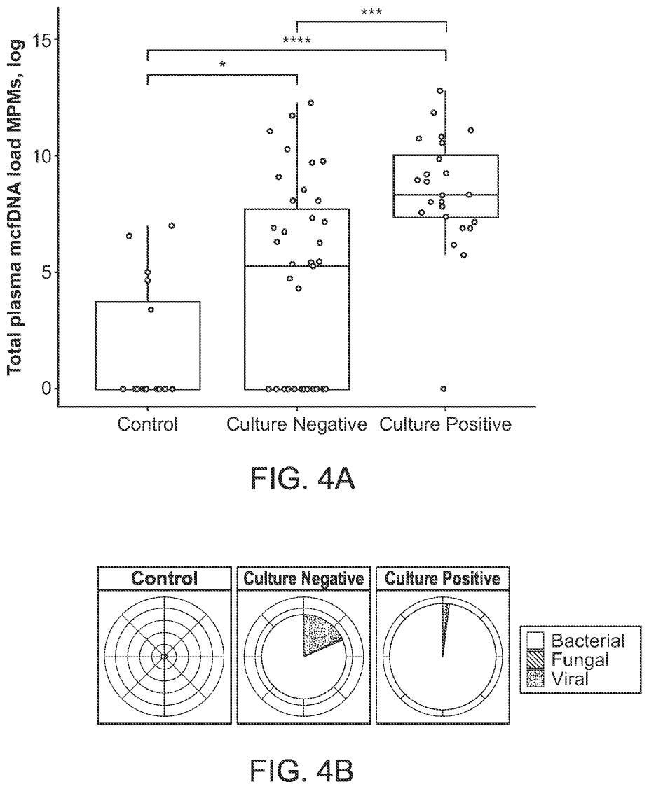

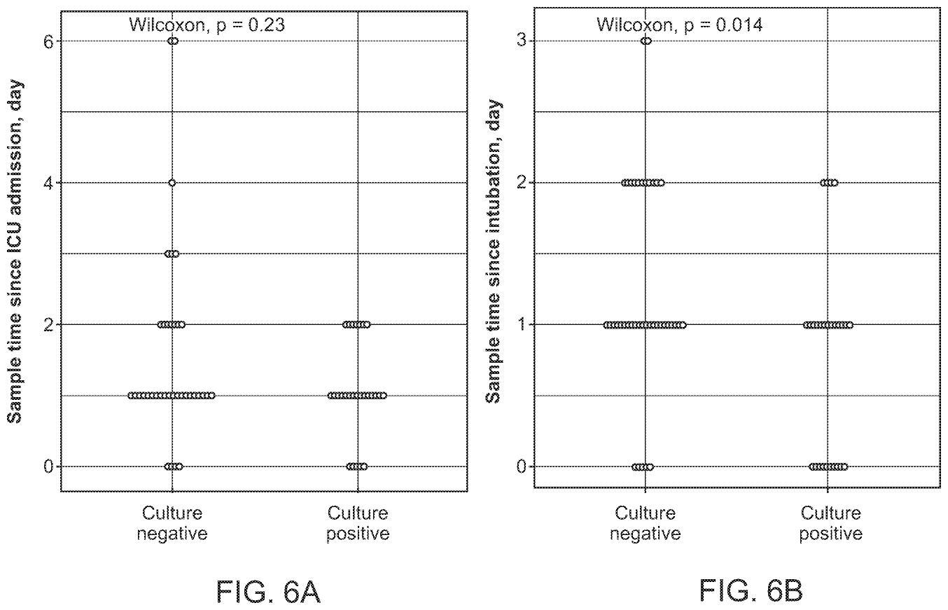

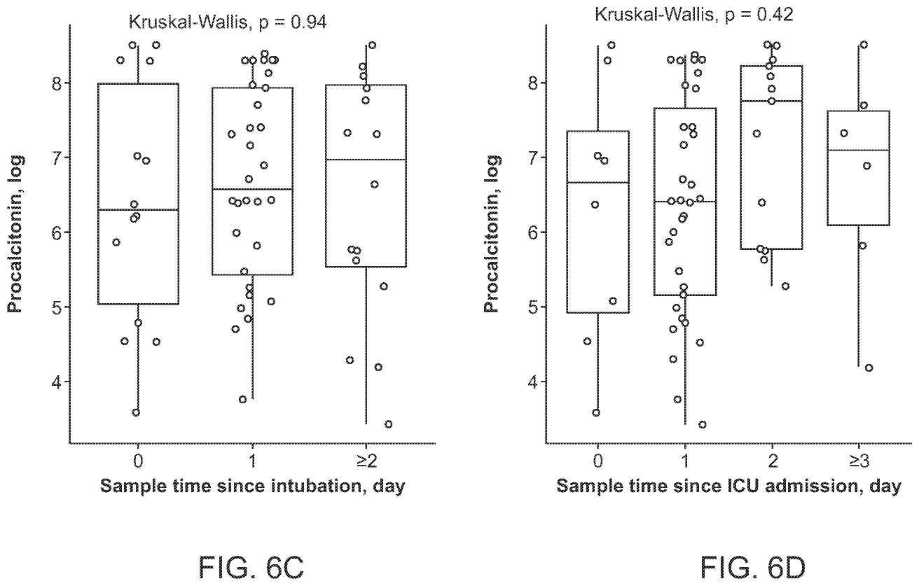

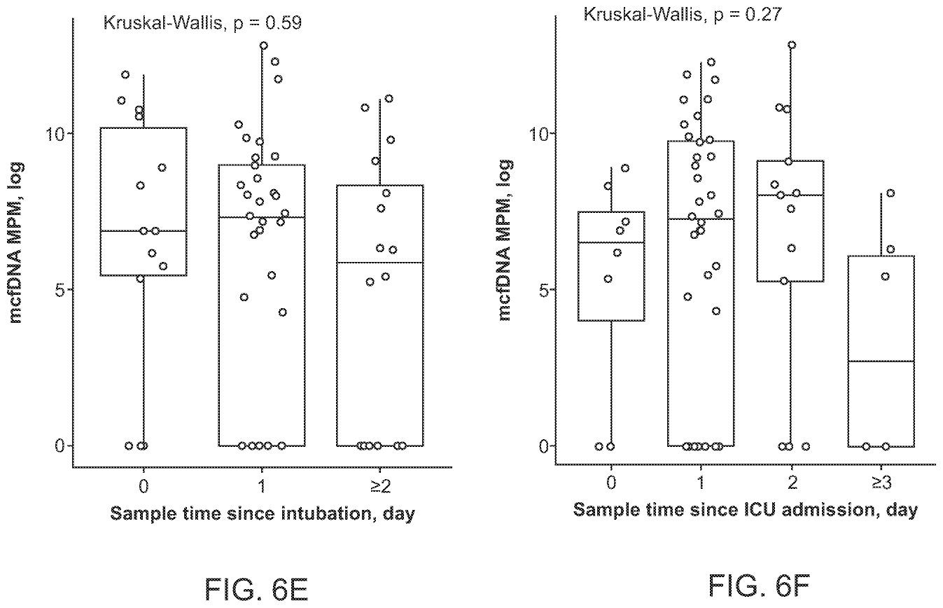

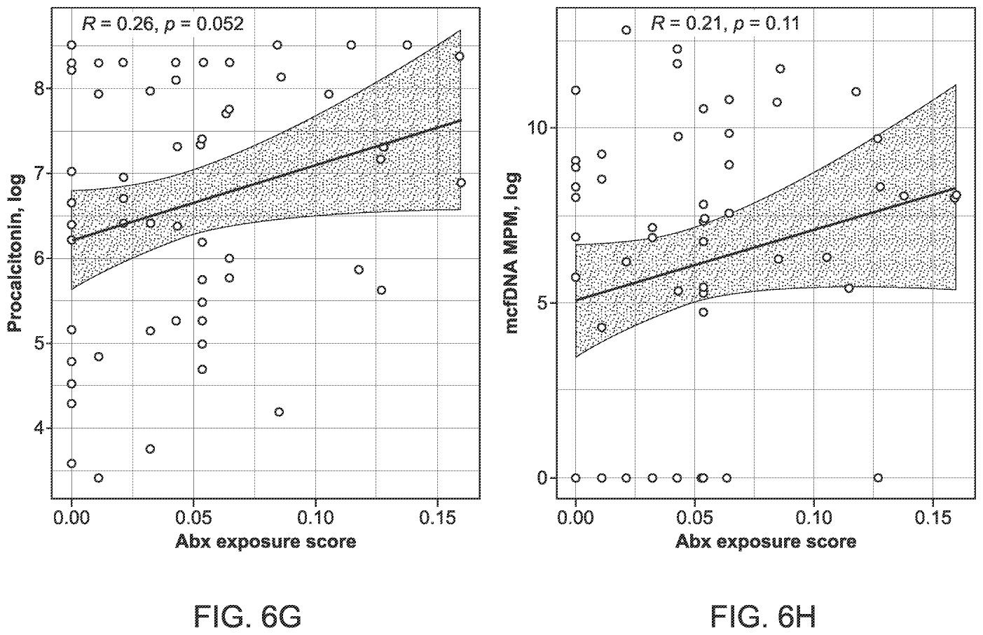

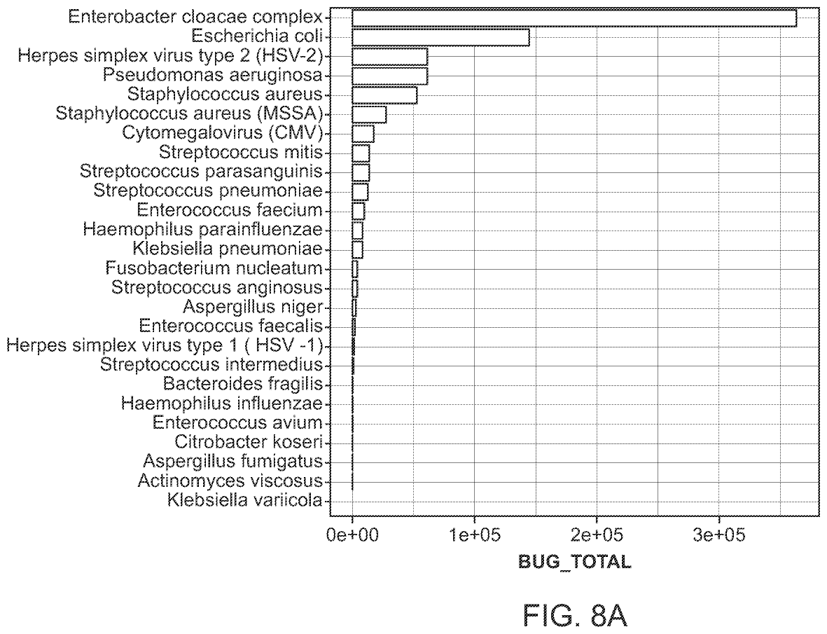

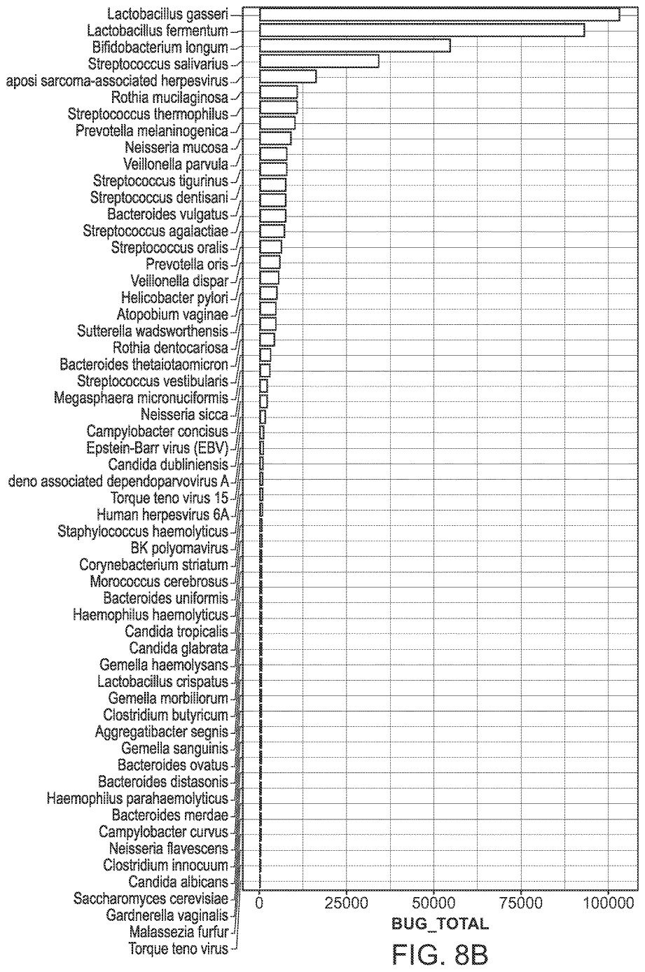

Patients with pneumonia (culture-positive or negative) had fewer ventilator-free days, higher CPIS, RSI, and levels of inflammatory biomarkers compared to controls (Table 1, p<0.05). Culture-positive patients had higher circulating mcfDNA compared to other groups (post-hoc p<0.001, FIG. 4A). Of the twenty-four culture-positive patients with detectable mcfDNA in plasma, only three (13%) were bacteremic. The majority (92%) of all detected mcfDNA sequences belonged to bacteria (FIG. 4B) and 64% of sequences were assigned to established respiratory pathogens (e.g., Staphylococcus aureus and Pseudomonas aeruginosa), See Table 2, which classifies recognized respiratory pathogens (with supporting literature, and list those of unclear clinical importance in the context of pneumonia. FIG. 4A shows plasma microbial cell-free DNA levels are elevated in culture-positive pneumonia compared with culture-negative pneumonia and uninfected controls and compared to culture-negative pneumonia patients (pairwise comparisons post hoc adjusted by Benjamini-Hochberg method). *, post hoc p<0.05; ***, post hoc p<0.005; ****, post hoc p<0.001. FIG. 4B shows the types of mcfDNA (bacterial, fungal, or viral) detected in culture-positive, culture-negative pneumonia and in uninfected controls depicted in pie charts. The radius of pie charts scales quadratically proportional to the sum of mcfDNA MPMs detected within each patient subgroup. The proportion of viral mcfDNA was significantly higher in the culture-negative (18.0%) compared to the culture-positive pneumonia (1.6%) group (p<0.0001 for z test of comparison of proportions). Loads of mcfDNA detected, by taxa, are visualised in FIG. 8. FIG. 8A and FIG. 8B show the sum of mcfDNA load detected across all participants by taxa, quantified as molecules per microliter (MPMs). FIG. 8A shows mcfDNA of recognized respiratory pathogen taxa; FIG. 8B shows mcfDNA of microbes with unclear clinical importance. A comparison between mcfDNA sequencing and culture results is shown in Table 3. Samples for mcfDNA sequencing were collected within 72 hours of intubation. No significant effect of timing of sample acquisition (from intubation or ICU admission) or intensity of antibiotic exposure prior to sampling on mcfDNA load was found (FIG. 6). FIG. 6A and FIG. 6B show the impact of timing of sampling and antibiotic exposure on mcfDNA and procalcitonin levels in patients with pneumonia. FIG. 6A shows time of sampling from ICU admission between culture positive and culture negative patients. FIG. 6B shows time of sampling from intubation between culture positive and culture negative patients. Culture-positive patients had relatively shorter time interval from intubation compared to culture-negative patients (p=0.014, Wilcoxon test). FIG. 6C and FIG. 6D shows procalcitonin levels did not differ by time of sampling from ICU admission (FIG. 6D) or intubation (FIG. 6C). FIG. 6E and FIG. F shows mcfDNA levels did not differ by time of sampling from ICU admission (FIG. 6F) or intubation (FIG. 6E). FIG. 6G and FIG. 6H shows procalcitonin (FIG. 6G) and mcfDNA levels (FIG. 6H) were not significantly associated with the antibiotic exposure score, applied as previously described. Kitsios 2020; Zhao, 2014, Sci Rep, 4:4345.

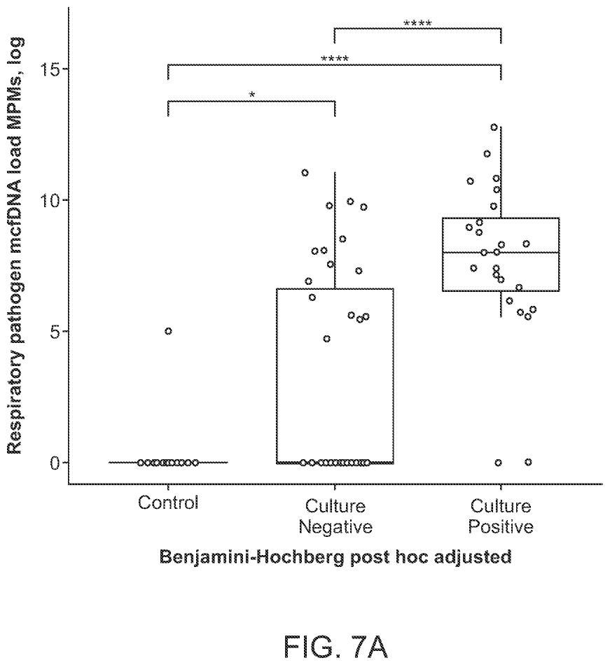

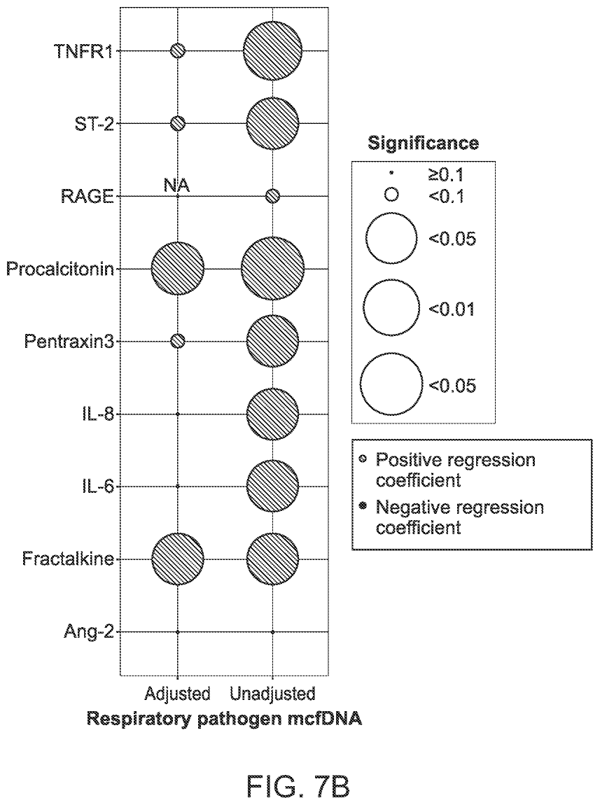

For host response, only pentraxin-3 was significantly elevated in the culture-positive vs. culture-negative participants among patients with pneumonia (post hoc p=0.05, Table 1). Linear regression models were built comparing plasma biomarkers (outcomes) to plasma mcfDNA levels (predictor) in unadjusted as well as adjusted models for a priori selected potential confounders. FIG. 7A shows culture-positive pneumonia patients had higher levels of plasma mcfDNA MPMs corresponding to recognized respiratory pathogens (Table 2) compared to culture-negative pneumonia patients, who in turn had also higher mcfDNA levels compared to uninfected controls (pairwise comparisons post hoc adjusted by Benjamini-Hochberg method). *, post hoc p<0.05; ****, post hoc p<0.001. FIG. 7B shows a graphical representation of linear regression models of plasma biomarkers (outcomes, shown in y-axis) against plasma mcfDNA levels of recognized respiratory pathogens (predictor, shown in x-axis) in unadjusted as well as adjusted models for a priori selected potential confounders, including (i) a surrogate of the microbial inoculum (culture-positive vs. negative classification), (ii) degree of lung injury (as depicted radiographically by RSI and by the epithelial injury biomarker receptor for advanced glycation end products-RAGE), and (iii) host innate immunity status (age, chronic obstructive pulmonary disease and immunosuppression).

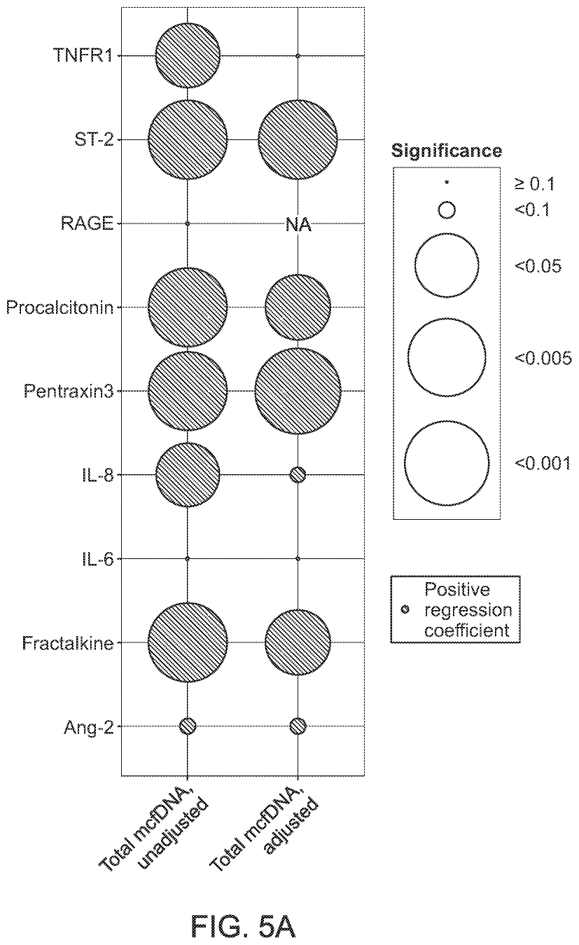

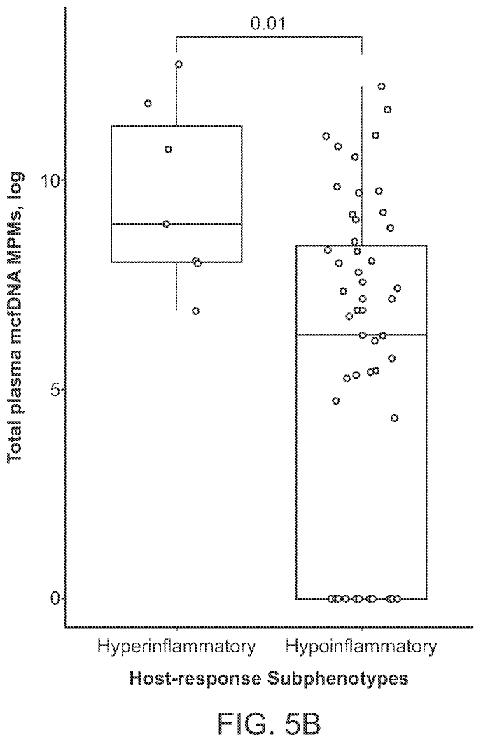

Table 4 reports the results for each regression model of calculations of estimated regression coefficients, 95% confidence intervals, and p values for significance of mcfDNA vs. plasma inflammatory biomarkers. Analyses were done for total mcfDNA, as well as for mcfDNA corresponding to recognized respiratory pathogens. All mcfDNA MPMs and biomarker measurements were log transformed; regression models with p<0.05 are shown in bold. In univariate linear regression models of host-response biomarkers against mcfDNA in patients with pneumonia, significant associations were detected for fractalkine, interleukin-8, procalcitonin, pentraxin-3, suppression of tumorigenicity-2 (ST-2), and soluble tumor necrosis factor receptor-1 (TNFR-1) levels (all p<0.05, FIG. 5A and Table 4). In multivariate regression models, the associations for fractalkine, procalcitonin, pentraxin-3 and ST-2 remained statistically significant (FIG. 5A and Table 4), suggesting independent effects of circulating mcfDNA on inflammatory responses. Patients with pneumonia assigned to the adverse hyper-inflammatory sub-phenotype (n=9, 13%) had significantly higher mcfDNA levels compared to hypo-inflammatory patients (p<0.01, FIG. 5B). FIG. 5A and FIG. 5B show circulating mcfDNA is associated with host inflammatory responses in patients with pneumonia. FIG. 5A is a graphical representation of linear regression models of plasma biomarkers (outcomes, shown in y-axis) against plasma mcfDNA levels (predictor, shown in x-axis) in unadjusted as well as adjusted models for a priori selected potential confounders, including (i) a surrogate of the microbial inoculum (culture-positive vs. negative classification), (ii) degree of lung injury (as depicted radiographically by RSI and by the epithelial injury biomarker receptor for advanced glycation end products-RAGE), and (iii) host innate immunity status (age, chronic obstructive pulmonary disease and immunosuppression). The direction of the effect size and corresponding statistical significance for the regression coefficient of mcfDNA on each plasma biomarker are visually presented by color and size coding, respectively; regression results are listed in detail in Table 4. FIG. 5B is a graph of host-response sub-phenotypes. Patients with pneumonia assigned to the hyperinflammatory sub-phenotype had significantly higher mcfDNA compared to hypo-inflammatory patients (median 7,731, interquartile range-IQR, MPMs, [3,100-79,849] vs. 546 [0-4,609] respectively, p<0.05). We assigned patients to the hyper- vs. hypo-inflammatory sub-phenotype based on a parsimonious predictive model utilizing levels of angiopoietin-2, procalcitonin, TNFR1 and bicarbonate.

The results revealed a novel link between circulating mcfDNA and systemic inflammation in patients with severe pneumonia, suggesting a biological microbe-host interaction in the systemic circulation. Circulating mcfDNA was associated with the intensified inflammatory host-responses, which have been reproducibly associated with worse clinical outcomes in severe pneumonia. Kitsios 2019. The discovery of a higher mcfDNA load in patients assigned to the hyperinflammatory sub-phenotype also linked microbiota and patient-level outcomes. [00159]). McfDNA of respiratory pathogens were detected in 82% and 38% of culture-positive and -negative patients, respectively. Table 2. Of these, one or more previously identified pneumonia pathogens were found in 12/18 (67%) of critically ill patients with pneumonia.

Notably, the significant associations between mcfDNA and fractalkine, procalcitonin, pentraxin-3 and ST-2 were independent of our radiographic (RSI) and biomarker (RAGE) measurements of the degree of lung injury. Microbial DNA is an established pathogen-associated molecular pattern (PAMP) that can stimulate pattern recognition receptors (PRRs) in innate immune cells to activate downstream inflammatory signaling See, e.g., Mogensen, 2009, Clin Microbiol Rev, 22:240-73.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

TABLE 1
Baseline characteristics, host response biomarkers and outcomes by clinical diagnosis.

| | | | | | | | P value for | P value for | P value for |
|---|---|---|---|---|---|---|---|---|---|
| | | | | | | | comparisons | culture- | comparisons |
| | | | | | | | between all 3 | positive vs | between |
| | | | Culture- | | Culture- | | subgroups, | negative | pneumonia |
| | Uninfected | | negative | | positive | | Kruskal- | pneumonia, | vs controls, |
| | controls^{#} | | pneumonia | | pneumonia | | Wallis/Fisher's | KW/Fishers' | Wilcoxon/ |
| | (n = 16) | | (n = 40) | | (n = 27) | | exact test | test, post hoc | Fisher's test |
| Age (median [IQR], | 56.2 [44.2, | | 54.8 [41.7, | | 56.0 [51.1, | | 0.9800 | 0.9741 | 0.9220 |
| yrs) | 69.4] | | 70.4] | | 62.3] | | | | |
| Male, N (%) | 12 | (75.0) | 21 | (52.5) | 19 | (70.4) | 0.1910 | 0.3080 | 0.3890 |
| COPD, N (%) | 1 | (6.2) | 5 | (12.5) | 8 | (29.6) | 0.1180 | 0.1820 | 0.2850 |
| Immunosuppression, | 3 | (18.8) | 6 | (15.0) | 9 | (33.3) | 0.3460 | 0.4050 | 1.0000 |
| N (%) | | | | | | | | | |
| BMI (median [IQR], | 27.7 [23.3, | | 29.8 [26.6, | | 25.6 [22.2, | | 0.0478 | 0.0525 | 0.5110 |
| kg · m⁻²) | 29.7] | | 35.1] | | 32.1] | | | | |
| CPIS (median [IQR]) | 2.0 [1.0, | | 6.0 [5.8, | | 8.0 [7.0, | | <0.0001 | 0.0260 | <0.0001 |
| | 4.0] | | 7.0] | | 8.0] | | | | |
| RSI (median [IQR]) | 4.0 [1.5, | | 33.5 [20.3, | | 18.0 [8.8, | | 0.0001 | 0.0075 | 0.0015 |
| | 15.5] | | 49.8] | | 27.0] | | | | |
| SOFA score^{$} | 5.0 [3.0, | | 6.0 [5.0, | | 6.0 [4.0, | | 0.3440 | 0.7180 | 0.1880 |
| (median [IQR]) | 7.5] | | 8.0] | | 8.0] | | | | |
| VFD (median [IQR], | 24.0 [21.8, | | 18.5 [0.0, | | 17.0 [8.5, | | 0.0029 | 0.3913 | 0.0009 |
| days) | 25.3] | | 24.0] | | 22.5] | | | | |
| Mortality at 30 days, | 1 | (6.2) | 10 | (25.0) | 5 | (18.5) | 0.3330 | 0.7660 | 0.1780 |
| N (%) | | | | | | | | | |
| Hypoinflammatory | 15 | (93.8) | 37 | (94.9) | 20 | (74.1) | 0.0480 | 0.0780 | 0.6780 |
| subphenotype, N (%) | | | | | | | | | |
| Plasma biomarkers, | | | | | | | | | |
| pg · mL⁻¹ | | | | | | | | | |
| Procalcitonin | 218.5 [80.8, | | 596.8 [187.5, | | 1496.4 [444.5, | | 0.0037 | 0.0746 | 0.0046 |
| (median [IQR]) | 358.6] | | 1787.9] | | 4040.7] | | | | |
| Pentraxin3 | 1068.8 [578.1, | | 3128.1 [1596.6, | | 6873.9 [3019.0, | | 0.0007 | 0.0485 | 0.0011 |
| (median [IQR]) | 2446.0] | | 6594.2] | | 11486.7] | | | | |
| IL-6 (median [IQR]) | 24.6 [16.2, | | 30.8 [10.5, | | 85.1 [31.1, | | 0.0537 | 0.0659 | 0.2490 |
| | 50.6] | | 80.7] | | 184.0] | | | | |
| IL-8 (median [IQR]) | 10.7 [6.8, | | 13.4 [7.0, | | 23.7 [10.9, | | 0.0382 | 0.1520 | 0.0500 |
| | 13.9] | | 23.4] | | 43.0] | | | | |
| Ang2 (median [IQR]) | 4030.3 [2298.1, | | 4859.6 [3227.3, | | 8662.1 [3547.6, | | 0.0163 | 0.4030 | 0.0061 |
| | 4667.2] | | 10960.4] | | 13471.2] | | | | |
| ST-2 (median [IQR]) | 46629.3 [22641.9, | | 121137.3 [52038.5, | | 177396.6 [100668.0, | | 0.0052 | 0.1770 | 0.0032 |
| | 149493.9] | | 282624.9] | | 578534.7] | | | | |
| RAGE (median [IQR]) | 1688.9 [1346.2, | | 2691.8 [1652.1, | | 4356.4 [2330.7, | | 0.0040 | 0.0864 | 0.0045 |
| | 3205.4] | | 5523.3] | | 6977.7] | | | | |
| TNFR1 (median [IQR]) | 2398.7 [1300.8, | | 2699.0 [2164.8, | | 4597.8 [2348.8, | | 0.0403 | 0.2240 | 0.0262 |
| | 3336.2] | | 4964.9] | | 9159.1] | | | | |
| Fractalkine (median | 841.4 [410.1, | | 1305.2 [410.1, | | 1591.3 [937.8, | | 0.0434 | 0.2180 | 0.0292 |
| [IQR]) | 1231.9] | | 2232.3] | | 2779.6] | | | | |
| McfDNA Sequencing | | | | | | | | | |
| Results | | | | | | | | | |
| Total plasma mcfDNA | 0.0 [0.0, | | 194.0 [0.0, | | 4085.0 [1553.0, | | <0.0001 | 0.0002 | 0.0003 |
| (median [IQR], MPM) | 48.5] | | 2343.5] | | 23663.2] | | | | |
| Bacterial mcfDNA | 0.0 [0.0, | | 0.0 [0.0, | | 4085.0 [1204.2 | | <0.0001 | <0.0001 | 0.0033 |
| (median [IQR]) | 7.5] | | 762.0] | | 17180.2] | | | | |
| Viral mcfDNA | 0.0 [0.0, | | 0.0 [0.0, | | 0.0 [0.0, | | 0.1630 | 0.1860 | 0.2830 |
| (median [IQR], MPM) | 0.0] | | 0.0] | | 0.0] | | | | |
| Fungal mcfDNA | 0.0 [0.0, | | 0.0 [0.0, | | 0.0 [0.0, | | 0.0808 | 0.1410 | 0.1360 |
| (median [IQR], MPM) | 0.0] | | 0.0] | | 0.0] | | | | |
| Presence of plasma | 1 | (6.2) | 15 | (37.5) | 22 | (81.5) | <0.0001 | 0.0005 | 0.0003 |

| | | | | | | |
|---|---|---|---|---|---|---|
| respiratory pathogen | | | | | | |
| mcfDNA, N (%) | | | | | | |
| Plasma respiratory | 0.0 [0.0, | 0.0 [0.0, | 3012.0 [702.0, | <0.0001 | <0.0001 | 0.0002 |
| pathogen mcfDNA | 0.0] | 762.0] | 11453.0] | | | |
| (median [IQR], MPM) | | | | | | |

^{$}= SOFA score calculation did not include the neurologic component, as all patients were intubated and receiving sedative medications, which impaired our ability to perform assessment of Glasgow Coma Scale in a consistent and reproducible manner.
Abbreviations: COPD, chronic obstructive pulmonary disease; BMI, body mass index; VFD, ventilator free day; CPIS, clinical pulmonary infection score; RAGE, receptor for advanced glycation end products; RSI, radiologic severity index; SOFA, sequential organ failure assessment; IL, interleukin; ST-2, suppression of tumorgenicity-2; TNFR-1, tumor necrosis factor receptor-1; mcfDNA, microbial cell-free DNA; MPM, microbial cell-free DNA per microliter of plasma.

TABLE 2
Microbes identified by mcfDNA sequencing

| Species | Category |
|---|---|
| Actinomyces viscosus | Recognized |
| | Respiratory Pathogen |
| Aspergillus fumigatus | Recognized |
| | Respiratory Pathogen |
| Aspergillus niger | Recognized |
| | Respiratory Pathogen |
| Bacteroides fragilis | Recognized |
| | Respiratory Pathogen |
| Citrobacter koseri | Recognized |
| | Respiratory Pathogen |
| Cytomegalovirus (CMV) | Recognized |
| | Respiratory Pathogen |
| Enterobacter cloacae complex | Recognized |
| | Respiratory Pathogen |
| Enterococcus avium | Recognized |
| | Respiratory Pathogen |
| Enterococcus faecalis | Recognized |
| | Respiratory Pathogen |
| Enterococcus faecium | Recognized |
| | Respiratory Pathogen |
| Escherichia coli | Recognized |
| | Respiratory Pathogen |
| Fusobacterium nucleatum | Recognized |
| | Respiratory Pathogen |
| Haemophilus influenzae | Recognized |
| | Respiratory Pathogen |
| Haemophilus parainfluenzae | Recognized |
| | Respiratory Pathogen |
| Herpes simplex virus type 1 (HSV-1) | Recognized |
| | Respiratory Pathogen |
| Herpes simplex virus type 2 (HSV-2) | Recognized |
| | Respiratory Pathogen |
| Klebsiella michiganensis | Recognized |
| | Respiratory Pathogen |
| Klebsiella pneumoniae | Recognized |
| | Respiratory Pathogen |
| Klebsiella variicola | Recognized |
| | Respiratory Pathogen |
| Porphyromonas gingivalis | Recognized |
| | Respiratory Pathogen |
| Pseudomonas aeruginosa | Recognized |
| | Respiratory Pathogen |
| Staphylococcus aureus | Recognized |
| | Respiratory Pathogen |
| Staphylococcus aureus (MSSA) | Recognized |
| | Respiratory Pathogen |
| Streptococcus anginosus | Recognized |
| | Respiratory Pathogen |
| Streptococcus intermedius | Recognized |
| | Respiratory Pathogen |
| Streptococcus mitis | Recognized |
| | Respiratory Pathogen |
| Streptococcus parasanguinis (AKA | Recognized |
| Streptococcus parasanguinius) | Respiratory Pathogen |
| Streptococcus pneumoniae | Recognized |
| | Respiratory Pathogen |
| Adeno-associated dependoparvovirus A | Microbe with unclear |
| | clinical importance |
| Aggregatibacter segnis | Microbe with unclear |
| | clinical importance |
| Atopobium vaginae | Microbe with unclear |
| | clinical importance |
| Bacteroides distasonis | Microbe with unclear |
| | clinical importance |
| Bacteroides merdae | Microbe with unclear |
| | clinical importance |
| Bacteroides ovatus | Microbe with unclear |
| | clinical importance |
| Bacteroides thetaiotaomicron | Microbe with unclear |
| | clinical importance |
| Bacteroides uniformis | Microbe with unclear |
| | clinical importance |
| Bacteroides vulgatus | Microbe with unclear |
| | clinical importance |
| Bifidobacterium longum | Microbe with unclear |
| | clinical importance |
| BK polyomavirus | Microbe with unclear |
| | clinical importance |
| Campylobacter concisus | Microbe with unclear |
| | clinical importance |
| Campylobacter curvus | Microbe with unclear |
| | clinical importance |
| Candida albicans | Microbe with unclear |
| | clinical importance |
| Candida dubliniensis | Microbe with unclear |
| | clinical importance |
| Candida glabrata | Microbe with unclear |
| | clinical importance |
| Candida tropicalis | Microbe with unclear |
| | clinical importance |
| Clostridium butyricum | Microbe with unclear |
| | clinical importance |
| Clostridium innocuum | Microbe with unclear |
| | clinical importance |
| Corynebacterium striatum | Microbe with unclear |
| | clinical importance |
| Epstein-Barr virus (EBV) | Microbe with unclear |
| | clinical importance |
| Gardnerella vaginalis | Microbe with unclear |
| | clinical importance |
| Gemella haemolysans | Microbe with unclear |
| | clinical importance |
| Gemella morbillorum | Microbe with unclear |
| | clinical importance |
| Gemella sanguinis | Microbe with unclear |
| | clinical importance |
| Haemophilus haemolyticus | Microbe with unclear |
| | clinical importance |
| Haemophilus parahaemolyticus | Microbe with unclear |
| | clinical importance |
| Helicobacter pylori | Microbe with unclear |
| | clinical importance |
| Human herpesvirus 6A | Microbe with unclear |
| | clinical importance |
| Human herpesvirus 6B | Microbe with unclear |
| | clinical importance |
| Kaposi sarcoma-associated herpesvirus | Microbe with unclear |
| | clinical importance |
| Lactobacillus crispatus | Microbe with unclear |
| | clinical importance |
| Lactobacillus fermentum | Microbe with unclear |
| | clinical importance |
| Lactobacillus gasseri | Microbe with unclear |
| | clinical importance |
| Malassezia furfur | Microbe with unclear |
| | clinical importance |
| Megasphaera micronuciformis | Microbe with unclear |
| | clinical importance |
| Morococcus cerebrosus | Microbe with unclear |
| | clinical importance |
| Neisseria flavescens | Microbe with unclear |
| | clinical importance |
| Neisseria mucosa | Microbe with unclear |
| | clinical importance |
| Neisseria sicca | Microbe with unclear |
| | clinical importance |
| Prevotella melaninogenica | Microbe with unclear |
| | clinical importance |
| Prevotella oris | Microbe with unclear |
| | clinical importance |
| Rothia dentocariosa | Microbe with unclear |
| | clinical importance |
| Rothia mucilaginosa | Microbe with unclear |
| | clinical importance |
| Saccharomyces cerevisiae | Microbe with unclear |
| | clinical importance |
| Staphylococcus haemolyticus | Microbe with unclear |
| | clinical importance |
| Streptococcus agalactiae | Microbe with unclear |
| | clinical importance |
| Streptococcus dentisani | Microbe with unclear |
| | clinical importance |
| Streptococcus oralis | Microbe with unclear |
| | clinical importance |
| Streptococcus salivarius | Microbe with unclear |
| | clinical importance |
| Streptococcus thermophilus | Microbe with unclear |
| | clinical importance |
| Streptococcus tigurinus | Microbe with unclear |
| | clinical importance |
| Streptococcus vestibularis | Microbe with unclear |
| | clinical importance |
| Sutterella wadsworthensis | Microbe with unclear |
| | clinical importance |
| Torque teno virus | Microbe with unclear |
| | clinical importance |
| Veillonella dispar | Microbe with unclear |
| | clinical importance |
| Veillonella parvula | Microbe with unclear |
| | clinical importance |

TABLE 3
A comparison between respiratory and blood culture results and plasma mcfDNA sequencing.

| Subject | Clinical | | | |
|---|---|---|---|---|
| ID | subgroup | McfDNA Sequencing Summary (bug, MPMs) | Respiratory Cultures | Blood Cultures |
| 4490 | control | Escherichia coli, 150 | N/A | No Growth 5 |
| | | | | Days |
| 4498 | control | No Organism Detected, 0 | N/A | No Growth 5 |
| | | | | Days |
| 4506 | control | No Organism Detected, 0 | N/A | N/A |
| 4531 | control | No Organism Detected, 0 | N/A | No Growth 5 |
| | | | | Days |
| 4533 | control | No Organism Detected, 0 | Moderate Normal | No Growth 5 |
| | | | oropharyngeal flora | Days |
| 4595 | control | Helicobacter pylori, 104 | N/A | N/A |
| 4671 | control | No Organism Detected, 0 | Moderate NRF | No Growth 5 |
| | | | | Days |
| 4729 | control | No Organism Detected, 0 | N/A | No Growth 5 |
| | | | | Days |
| 4751 | control | No Organism Detected, 0 | Heavy Klebsiella species, | No Growth 5 |
| | | | Light NRF | Days |
| 4759 | control | No Organism Detected, 0 | N/A | No Growth 5 |
| | | | | Days |
| 4760 | control | No Organism Detected, 0 | Rare NRF | No Growth 5 |
| | | | | Days |
| 4761 | control | No Organism Detected, 0 | N/A | N/A |
| 4763 | control | No Organism Detected, 0 | N/A | N/A |
| 4773 | control | Prevotella melaninogenica, 437, Veillonella dispar, 146, | No growth | No Growth 5 |
| | | Rothia mucilaginosa, 123 | | Days |
| 4798 | control | Prevotella melaninogenica, 30 | N/A | N/A |
| 4828 | control | Epstein-Barr virus (EBV), 1106 | N/A | No Growth 5 |
| | | | | Days |
| 4502 | cxneg | Escherichia coli, 546 | Rare Normal | No Growth 5 |
| | | | oropharyngeal flora | Days |
| 4563 | cxneg | Enterococcus faecium, 1154, Torque teno virus 15, 892, | No growth | No Growth 5 |
| | | Candida glabrata, 340, Streptococcus mitis, 330, | | Days |
| | | Bacteroides vulgatus, 259, Streptococcus salivarius, 185 | | |
| 4566 | cxneg | No Organism Detected, 0 | No growth for 2 days | No Growth 5 |
| | | | | Days |
| 4576 | cxneg | No Organism Detected, 0 | NRF | No Growth 5 |
| | | | | Days |
| 4604 | cxneg | Lactobacillus fermentum, 37296, Streptococcus salivarius, | No growth | No Growth 5 |
| | | 25570, Streptococcus parasanguinis, 8492, Rothia | | Days |
| | | mucilaginosa, 8429, Veillonella parvula, 7695, Prevotella | | |
| | | melaninogenica, 7084, Haemophilus parainfluenzae, 6695, | | |
| | | Veillonella dispar, 4772, Rothia dentocariosa, 4716, | | |
| | | Megasphaera micronuciformis, 2990, Neisseria sicca, 2044, | | |
| | | Campylobacter concisus, 2008, Escherichia coli, 1561, | | |
| | | Fusobacterium nucleatum, 1315 | | |
| 4615 | cxneg | Epstein-Barr virus (EBV), 224 | Normal oropharyngeal | No Growth 5 |
| | | | flora | Days |
| 4619 | cxneg | No Organism Detected, 0 | Rare Normal | No Growth 5 |
| | | | oropharyngeal flora | Days |
| 4621 | cxneg | Prevotella melaninogenica, 1287, Veillonella dispar, 151, | No growth | No Growth 5 |
| | | Campylobacter concisus, 89 | | Days |
| 4639 | cxneg | Morococcus cerebrosus, 142, Veillonella parvula, 68 | Rare NRF | No Growth 5 |
| | | | | Days |
| 4649 | cxneg | Streptococcus pneumoniae, 8537, Klebsiella pneumoniae, | Rare NRF | No Growth |
| | | 8179, Herpes simplex virus type 1 (HSV-1), 486 | | |
| 4658 | cxneg | Pseudomonas aeruginosa, 1920, Candida dubliniensis, | No growth | No Growth 5 |
| | | 1062, Rothia mucilaginosa, 195, Torque teno virus, 42 | | Days |
| 4688 | cxneg | Helicobacter pylori, 5451, Streptococcus pneumoniae, 2957, | NRF | No Growth 5 |
| | | Citrobacter koseri, 169, Haemophilus haemolyticus, 169 | | Days |
| 4693 | cxneg | Candida dubliniensis, 74 | No Growth 2 Days | No Growth 5 |
| | | | | Days |
| 4697 | cxneg | Escherichia coli, 978 | Light NRF | No Growth 5 |
| | | | | Days |
| 4698 | cxneg | No Organism Detected, 0 | N/A | No Growth 5 |
| | | | | Days |
| 4711 | cxneg | Human herpesvirus 6A, 844 | NRF | No Growth 5 |
| | | | | Days |
| 4742 | cxneg | No Organism Detected, 0 | Rare NRF | N/A |
| 4743 | cxneg | No Organism Detected, 0 | N/A | N/A |
| 4745 | cxneg | Kaposi sarcoma-associated herpesvirus, 16123, | Heavy NRF, Rare GNR | No Growth 5 |
| | | Cytomegalovirus (CMV), 273 (NOTE: Kaposi sarcoma | | Days |
| | | diagnosed weeks later with Biopsy) | | |
| 4746 | cxneg | No Organism Detected, 0 | No Growth 2 Days | No Growth 5 |
| | | | | Days |
| 4748 | cxneg | No Organism Detected, 0 | Rare NRF | No Growth 5 |
| | | | | Days |
| 4753 | cxneg | Cytomegalovirus (CMV), 5113 | Light NRF | No Growth 5 |
| | | | | Days |
| 4754 | cxneg | Bacteroides vulgatus, 194 | No Growth 2 Days | No Growth 5 |
| | | | | Days |
| 4755 | cxneg | BK polyomavirus, 522 | No growth | N/A |
| 4780 | cxneg | Escherichia coli, 114 | NRF | No Growth 5 |
| | | | | Days |
| 4793 | cxneg | No Organism Detected, 0 | No growth | No Growth 5 |
| | | | | Days |
| 4794 | cxneg | No Organism Detected, 0 | NRF | No Growth 5 |
| | | | | Days |
| 4804 | cxneg | No Organism Detected, 0 | No growth | No Growth 5 |
| | | | | Days |
| 4805* | cxneg | Lactobacillus gasseri, 102949, Lactobacillus fermentum, | Heavy NRF | Lactobacillus |
| | | 55713, Streptococcus mitis, 8674, Streptococcus tigurinus, | | gasseri |
| | | 7663, Streptococcus dentisani, 7435, Streptococcus oralis, | | (considered |
| | | 7093, Streptococcus salivarius, 4709, Streptococcus | | contaminant |
| | | parasanguinis, 4592, Streptococcus anginosus, 4362, | | by clinical |
| | | Escherichia coli, 3412, Streptococcus vestibularis, 3207, | | team) |
| | | Saccharomyces cerevisiae, 96 | | |
| 4807 | cxneg | No Organism Detected, 0 | Moderate NRF | No Growth 5 |
| | | | | Days |
| 4808 | cxneg | Herpes simplex virus type 2 (HSV-2), 61551, Adeno- | Light NRF, Yeast | No Growth 5 |
| | | associated dependoparvovirus A, 977 | | Days; No |
| | | | | Fungus |
| | | | | isolated 28 |
| | | | | Days |
| 4830 | cxneg | Pseudomonas aeruginosa, 233 | Light NRF | No Growth 5 |
| | | | | Days |
| 4835 | cxneg | No Organism Detected, 0 | N/A | No Growth 5 |
| | | | | Days |
| 4839 | cxneg | No Organism Detected, 0 | NRF | No Growth 5 |
| | | | | Days |
| 4845 | cxneg | Streptococcus salivarius, 448, Streptococcus parasanguinis, | Heavy NRF | No Growth 5 |
| | | 253, Bacteroides distasonis, 169, Corynebacterium striatum, | | Days |
| | | 151, Bacteroides thetaiotaomicron, 134, Bacteroides | | |
| | | merdae, 125 | | |
| 4847 | cxneg | Haemophilus influenzae (Sequencing passed qualitatively) | No Growth 2 Days | No Growth 5 |
| | | | | Days |
| 4848 | cxneg | No Organism Detected, 0 | No Growth 2 Days | No Growth 5 |
| | | | | Days |
| 4850 | cxneg | No Organism Detected, 0 | N/A | No Growth 5 |
| | | | | Days |
| 4860 | cxneg | No Organism Detected, 0 | N/A | No Growth 5 |
| | | | | Days |
| 4861 | cxneg | Streptococcus thermophilus, 10810, Neisseria mucosa, | N/A | Enterococcus |
| | | 9000, Streptococcus salivarius, 2818, Escherichia coli, 1841, | | faecalis |
| | | Haemophilus parainfluenzae, 1375, Rothia mucilaginosa, | | (considered |
| | | 1253, Veillonella dispar, 760, Prevotella melaninogenica, | | contaminant |
| | | 600, Bacteroides uniformis, 385 | | by clinical |
| | | | | team) |
| 4484 | cxpos | Streptococcus mitis, 2775, Prevotella melaninogenica, 318, | NRF | No Growth 5 |
| | | Haemophilus parainfluenzae, 262, Gemella haemolysans, | | Days |
| | | 213, Veillonella dispar, 163, Haemophilus parahaemolyticus, | | |
| | | 161, Haemophilus haemolyticus, 123 | | |
| 4507 | cxpos | Prevotella oris, 6145, Fusobacterium nucleatum, 3158, | Light Staphylococcus | No Growth 5 |
| | | Streptococcus intermedius, 944 | aureus, Light Normal | Days |
| | | | oropharyngeal flora | |
| 4509 | cxpos | Cytomegalovirus (CMV), 12144, Pseudomonas aeruginosa, | Heavy Stenotrophomonas | No Growth 5 |
| | | 4308, Enterococcus faecalis, 830, Staphylococcus | maltophilia, Heavy Normal | Days |
| | | haemolyticus, 800, Herpes simplex virus type 1 (HSV-1), | oropharyngeal flora | |
| | | 424, Rothia mucilaginosa, 385 | | |
| 4514 | cxpos | Staphylococcus aureus, 475 | Heavy Staphylococcus | No Growth 5 |
| | | | aureus | Days |
| 4544 | cxpos | Streptococcus pneumoniae, 777, Aggregatibacter segnis, | Heavy Streptococcus | No Growth 5 |
| | | 209 | pneumoniae, Heavy | Days |
| | | | Staphylococcus aureus | |
| | | | Confirmed Methicillin | |
| | | | Sensitive | |
| 4547* | cxpos | Staphylococcus aureus, 45941 | Heavy MRSA, Normal | Gram Positive |
| | | | oropharyngeal flora | Cocci in |
| | | | | clusters; |
| | | | | Staphylococcus |
| | | | | aureus; |
| 4586 | cxpos | Aspergillus niger, 2986 | Aspergillus fumigatus, | No Growth 5 |
| | | | Rare NRF | Days |
| 4588 | cxpos | Pseudomonas aeruginosa, 4155 | Moderate Staphylococcus | No Growth 5 |
| | | | aureus, Light NRF | Days |
| 4629 | cxpos | Escherichia coli, 6461, Bacteroides vulgatus, 751 | Light Escherichia coli | No Growth 5 |
| | | | | Days |
| 4630 | cxpos | Streptococcus agalactiae, 656, Haemophilus influenzae, | Light Group B | No Growth 5 |
| | | 347, Lactobacillus crispatus, 274 | Streptococci (S. | Days |
| | | | agalactiae), Light Normal | |
| | | | oropharyngeal flora | |
| 4652 | cxpos | Enterococcus faecalis, 1035, Morococcus cerebrosus, 348, | Moderate MRSA, | No Growth 5 |
| | | Corynebacterium striatum, 343, Veillonella parvula, 142, | Moderate NRF | Days |
| | | Neisseria sicca, 132, Campylobacter curvus, 116, | | |
| | | Staphylococcus aureus, 95, Actinomyces viscosus, 93, | | |
| | | Malassezia furfur, 85, Fusobacterium nucleatum, 84 | | |
| 4668 | cxpos | Streptococcus parasanguinius (Sequencing passed | Light Klebsiella species, | N/A |
| | | qualitatively) | NRF | |
| 4678 | cxpos | Pseudomonas aeruginosa, 49392, Aspergillus fumigatus, | Moderate Pseudomonas | N/A |
| | | 107 | aeruginosa, Aspergillus | |
| | | | fumigatus, Heavy Normal | |
| | | | oropharyngeal flora | |
| 4679 | cxpos | Pseudomonas aeruginosa, 1051, Bacteroides vulgatus, 414, | Light Pseudomonas | No Growth 5 |
| | | Bacteroides ovatus, 180 | aeruginosa | Days |
| 4687 | cxpos | QC failure during sequencing | Few Candida species, | No Growth 5 |
| | | | Few Alpha Hemolytic | Days |
| | | | Streptococci | |
| 4707 | cxpos | QC failure during sequencing | Heavy Staph aureus, | No Growth 5 |
| | | | Moderate NRF | Days |
| 4718 | cxpos | No Organism Detected, 0 | Light Klebsiella | No Growth 5 |
| | | | pneumoniae, Moderate | Days |
| | | | NRF | |
| 4726 | cxpos | Staphylococcus aureus (MSSA), 1375, Streptococcus mitis, | Heavy Staph aureus, Light | No Growth 5 |
| | | 123, Candida albicans, 101, Herpes simplex virus type 1 | NRF | Days |
| | | (HSV-1), 98, Gardnerella vaginalis, 93, Streptococcus | | |
| | | salivarius, 83, Klebsiella variicola, 59 | | |
| 4727 | cxpos | Streptococcus mitis, 1312, Candida tropicalis, 372, | Heavy Staph aureus, | No Growth 5 |
| | | Staphylococcus aureus, 305, Prevotella melaninogenica, | Moderate NRF | Days |
| | | 264, Gemella morbillorum, 228, Gemella sanguinis, 197, | | |
| | | Neisseria flavescens, 108, Haemophilus haemolyticus, 92, | | |
| | | Neisseria mucosa, 81, Gemella haemolysans, 77 | | |
| 4738 | cxpos | Enterobacter cloacae complex, 331992, Staphylococcus | Heavy Carbapenem | No Growth 5 |
| | | aureus (MSSA), 24360 | resistant Enterobacter | Days |
| | | | cloacae complex. | |
| | | | Confirmed as a | |
| | | | carbapenemase producer. | |
| | | | Moderate Staphylococcus | |
| | | | aureus | |
| 4741* | cxpos | Escherichia coli, 122894, Streptococcus agalactiae, 6735, | N/A | Gram Positive |
| | | Staphylococcus aureus, 5151, Bacteroides thetaiotaomicron, | | Cocci in |
| | | 4005 | | chains in |
| | | | | pairs; |
| | | | | Streptococcus |
| | | | | constellatus; |
| | | | | Staphylococcus |
| | | | | aureus |
| | | | | confirmed |
| | | | | methicillin |
| | | | | sensitive; |
| 4752 | cxpos | Enterobacter cloacae complex, 31429, Atopobium vaginae, | Light Proteus mirabilis, | No Growth 5 |
| | | 4946, Staphylococcus aureus (MSSA), 1605 | Moderate Staph | Days |
| | | | aureus(MRSA), NRF | |
| 4757 | cxpos | Escherichia coli, 6733, Bacteroides fragilis, 705, | Moderate E. Coli, | No Growth 5 |
| | | Enterococcus avium, 293 | Moderate Staph | Days |
| | | | aureus(MRSA), Light | |
| | | | Klebsiella pneumoniae, | |
| | | | Moderate Group B | |
| | | | Streptococci(S. | |
| | | | agalactiae), Light NRF | |
| 4770 | cxpos | Bacteroides vulgatus, 261, Clostridium butyricum, 215, | Rare MRSA, Light NRF | No Growth 5 |
| | | Enterobacter cloacae complex, 152, Lactobacillus gasseri, | | Days |
| | | 131, Escherichia coli, 113, Clostridium innocuum, 101 | | |
| 4771 | cxpos | Bifidobacterium longum, 54711, Bacteroides vulgatus, 5554, | MSSA, NRF | No Growth 5 |
| | | Sutterella wadsworthensis, 4777 | | Days |
| 4810* | cxpos | Streptococcus mitis, 307 | Heavy Staph aureus, | Staphylococcus |
| | | | Heavy Group B | aureus |
| | | | streptococci(S. | confirmed |
| | | | agalactiae), Light NRF | methicillin |
| | | | | sensitive; |
| | | | | Gram Positive |
| | | | | Cocci in |
| | | | | clusters |
| 4858 | cxpos | Enterococcus faecium, 8888, Staphylococcus aureus, 481, | N/A | No Growth 5 |
| | | Rothia mucilaginosa, 472 | | Days |

Abbreviations: N/A, no corresponding sample was acquired from the time span; *cases with bacteremia Cx, culture; MPM, mcfDNA molecules per microliter; MRSA, methicillin resistant Staphylococcus aureus; MSSA, methicillin sensitive S. aureus; neg, negative; NRF, normal respiratory flora; pos, positive.

TABLE 4
Linear regression results for mcfDNA and inflammatory biomarkers.
Total mcfDNA load

| | Univariate regression | | | Multivariate regression | | |
|---|---|---|---|---|---|---|
| Inflammation | coefficient (95% | | Univariate | coefficient (95% | | Multivariate |
| marker | Confident Interval) | | p value | Confident Interval) | | p value |
| ST-2 | 0.107 | (0.044, 0.169) | 0.0012 | 0.117 | (0.045, 0.190) | 0.0020 |
| IL-6 | 0.086 | (−0.021, 0.193) | 0.1123 | 0.040 | (−0.090, 0.169) | 0.5389 |
| IL-8 | 0.097 | (0.022, 0.173) | 0.0119 | 0.081 | (−0.013, 0.174) | 0.0881 |
| Ang-2 | 0.045 | (−0.003, 0.092) | 0.0677 | 0.050 | (−0.007, 0.107) | 0.0837 |
| Pentraxin3 | 0.125 | (0.048, 0.202) | 0.0020 | 0.162 | (0.071, 0.253) | 0.0008 |
| Procalcitonin | 0.132 | (0.048, 0.217) | 0.0027 | 0.129 | (0.032, 0.226) | 0.0102 |
| TNFR1 | 0.054 | (0.011, 0.098) | 0.0155 | 0.035 | (−0.007, 0.077) | 0.1027 |
| RAGE | 0.029 | (−0.022, 0.079) | 0.2596 | NA | | NA |
| Fractalkine | 0.069 | (0.023, 0.114) | 0.0036 | 0.069 | (0.011, 0.126) | 0.0199 |

Recognized respiratory pathogens mcfDNA load

| | Univariate regression | | | Multivariate regression | | |
|---|---|---|---|---|---|---|
| | coefficient (95% | | Univariate | coefficient (95% | | Multivariate |
| | Confident Interval) | | p value | Confident Interval) | | p value |
| ST-2 | 0.075 | (0.011, 0.138) | 0.0218 | 0.066 | (−0.010, 0.143) | 0.0880 |
| IL-6 | 0.105 | (0.003, 0.208) | 0.0447 | 0.047 | (−0.082, 0.176) | 0.4706 |
| IL-8 | 0.094 | (0.021, 0.168) | 0.0151 | 0.070 | (−0.024, 0.163) | 0.1428 |
| Ang-2 | 0.027 | (−0.02, 0.074) | 0.2606 | 0.025 | (−0.033, 0.083) | 0.3947 |
| Pentraxin3 | 0.087 | (0.009, 0.165) | 0.0296 | 0.095 | (−0.003, 0.192) | 0.0573 |
| Procalcitonin | 0.128 | (0.046, 0.210) | 0.0028 | 0.124 | (0.027, 0.221) | 0.0134 |
| TNFR1 | 0.056 | (0.014, 0.098) | 0.0097 | 0.037 | (−0.005, 0.079) | 0.0803 |
| RAGE | 0.045 | (−0.003, 0.094) | 0.0637 | NA | | NA |
| Fractalkine | 0.059 | (0.014, 0.104) | 0.0103 | 0.065 | (0.007, 0.122) | 0.0279 |

Abbreviations: Ang-2, angiopoietin-2; IL, interleukin; RAGE, receptor for advanced glycation end product; ST-2, suppression of tumorigenicity-2; TNFR-1, tumour necrosis factor receptor 1.

TABLE 5
Weighting score and antimicrobial spectrum classification for antibiotics
administered during hospitalization and prior to plasma sampling.
The antibiotic exposure was modeled with a published score (Han, 2006,
J Clin Microbiol, 44: 160-65) that considered dosing duration,
timing of administration and specific antibiotic type.

| | Weighting | Anti-bacterial |
|---|---|---|
| Antibiotic | score | spectrum category |
| Amikacin | 0.33 | Gram-negative |
| Amoxicillin-clavulanate | 0.5 | Broad-spectrum |
| Ampicillin-sulbactam | 0.5 | Broad-spectrum |
| Azithromycin | 0.17 | Atypicals |
| Cefazolin | 0.33 | Gram-positive |
| Cefepime | 0.5 | Broad-spectrum |
| Cefotaxime | 0.33 | Broad-spectrum |
| Ceftazidime | 0.33 | Broad-spectrum |
| Ceftriaxone | 0.33 | Broad-spectrum |
| Cefuroxime | 0.5 | Broad-spectrum |
| Cephalexin | 0.33 | Gram-positive |
| Ciprofloxacin | 0.5 | Broad-spectrum |
| Clarithromycin | 0.17 | Atypicals |
| Clindamycin | 0.17 | Anaerobes |
| Erythromycin | 0.17 | Atypicals |
| Gentamicin | 0.33 | Gram-negative |
| Imipenem-cilastatin | 0.5 | Broad-spectrum |
| Levofloxacin | 0.5 | Broad-spectrum |
| Linezolid | 0.33 | Gram-positive |
| Meropenem | 0.5 | Broad-spectrum |
| Metronidazole | 0.17 | Anaerobes |
| Nafcillin | 0.5 | Gram-positive |
| Piperacillin-tazobactam | 0.33 | Broad-spectrum |
| Tobramycin | 0.17 | Gram-negative |
| Trimethoprim-sulfamethoxazole | 0.33 | Gram-positive |
| Vancomycin | 0.33 | Gram-positive |

While preferred embodiments of the present invention have been shown and described herein, such embodiments are provided by way of example only. Numerous variations, changes, and substitutions are possible within the scope of the invention. It should be understood that various alternatives to the embodiments of the invention described herein may be employed in practicing the invention. It is intended that the following claims define the scope of the invention and that methods and structures within the scope of these claims and their equivalents be covered thereby.

1-94. (canceled)

95. A method of detecting a secondary infection in a human subject with a first infection, comprising:
(a) providing a first plasma sample from blood obtained from the subject with the first infection, wherein the plasma sample comprises microbial cell-free nucleic acids (mcfNA) from at least two different microbes;
(b) producing a sequencing library comprising mcfNA attached to adapters;
(c) measuring an amount of total mcfNA in the first plasma sample by performing massively parallel next generation sequencing on the sequencing library to obtain sequence reads comprising the mcfNA attached to adapters, wherein the total mcfNA comprises mcfNA from at least two different microbes;
(d) providing a second plasma sample from blood obtained from the subject with the first infection, wherein the second plasma sample comprises one or more inflammatory biomarkers;
(e) measuring an amount of the one or more inflammatory biomarkers in the second plasma sample;
(f) comparing the amount of total mcfNA comprising mcfNA from at least two different microbes to a threshold amount of total mcfNA;
(g) comparing the amount of the one or more inflammatory biomarkers to a threshold amount of inflammatory biomarkers; and
(h) detecting a secondary infection that is different from the first infection when the amount of total mcfNA comprising mcfNA from at least two different microbes exceeds the threshold amount of total mcfNA and wherein the amount of the one or more inflammatory biomarkers exceeds the threshold amount of inflammatory biomarkers.

96. The method of claim 95, wherein the mcfNA is microbial cell-free DNA (mcfDNA).

97. The method of claim 95, wherein the one or more inflammatory biomarkers are RNA biomarkers.

98. The method of claim 95, wherein the one or more inflammatory biomarkers are protein biomarkers.

99. The method of claim 98, wherein the protein biomarkers are detected using an immunoassay.

100. The method of claim 95, wherein the massively parallel next generation sequencing assay is a sequencing-by-synthesis assay.

101. The method of claim 95, wherein the one or more inflammatory biomarkers are at least one inflammatory biomarker selected from the group consisting of IL-6, IL-8, IL-10, RAGE, TNFR1, angiopoietin-2, procalcitonin, fractalkine, pentraxin-3, and ST2.

102. The method of claim 95, wherein the one or more inflammatory biomarkers comprise IL-8 or ST2.

103. The method of claim 95, further comprising administering a therapeutic drug to the human subject based on the detecting a secondary infection, wherein the therapeutic drug is an antimicrobial drug, an antibiotic drug, an antifungal drug, or any combination thereof.

104. The method of claim 95, wherein the first infection is a COVID-19 infection.

105. The method of 95, wherein the secondary infection is a bacterial or fungal infection.

106. The method of 95, wherein the first and secondary infections are respiratory infections caused by different microbes.

107. The method of claim 95, wherein the at least two microbes are at least two microbes from the group consisting of S. aureus, P. aeruginosa and K. pneumoniae.

108. The method of claim 95, wherein the first infection is culture-positive pneumonia.

109. The method of 95, wherein the first infection is culture-negative pneumonia.

110. The method of claim 95, wherein the at least two microbes comprise Candida.

111. The method of claim 95, wherein the amount of total mcfNA is an aggregated amount of total mcfNA from respiratory pathogens in the sample.

112. The method of claim 95, wherein the threshold amount of total mcfNA is an amount of mcfNA measured in plasma of a healthy or un-infected subject.

113. The method of claim 95, wherein the plasma or blood sample is spiked with a known concentration of synthetic normalization controls.

114. The method of claim 95, wherein the measuring the amount of mcfNA in the first plasma sample comprises:
(a) aligning the sequence reads with a microorganism database, wherein the microorganism database comprises a microorganism library comprising more than 10,000 genomic reference sequences;
(b) retaining reliable sequence reads comprising alignments with high percent identity and high query coverage;
(c) assigning relative abundances to each taxon based on the number of reliable sequence reads and their alignments;
(d) computing statistical significance values for each estimate of taxon abundance; and
(e) using taxon abundance to determine mcfNA concentration; and/or
(f) using abundance of spiked synthetic normalization controls to calculate the molecules per microliter (MPM) value of mcfNA in the sample.

115. The method of claim 114, wherein the amount of mcfNA is measured in molecules per microliter of plasma (MPM).

116. The method of claim 95, wherein the threshold amount of total mcfNA is greater than 400 MPM for all types of mcfNA in the sample.

117. The method of claim 95, wherein the threshold amount of total mcfNA is greater than 600 MPM for total mcfNA in the sample when the total mcfNA is determined by aligning sequence reads to a genomic database comprising sequences from at least 100 different microbes.

118. The method of claim 95, wherein the threshold amount of total mcfNA is greater than 4000 MPM for mcfNA from respiratory pathogens in the sample.

119. The method of claim 95, wherein the threshold amount of total mcfNA is greater than 4000 MPM when the total mcfNA is determined by aligning sequence reads to a genomic database comprising sequences from at least 100 different microbes.

120. The method of claim 95, wherein the subject in (a) has received an empiric antibiotic.

121. A method of treating an inflammatory response in a human patient, comprising:
(a) providing a plasma sample from the human patient;
(b) detecting an inflammatory response in the human patient when an amount of total mcfNA in the blood plasma sample comprises mcfNA from at least two different microbes and exceeds a threshold amount of total mcfNA; and
(c) administering an anti-inflammatory drug to the human patient to treat the inflammatory response.

122. The method of claim 121, wherein the human patient has a culture-negative pneumonia.

123. The method of claim 121, wherein the anti-inflammatory drug is a steroidal or a non-steroidal anti-inflammatory drug (NSAID).

124. The method of claim 121, wherein the detecting an inflammatory response comprises detecting one or more inflammatory biomarkers.

125. The method of claim 124, wherein the one or more inflammatory biomarkers are selected from the group consisting of IL-6, IL-8, IL-10, RAGE, TNFR1, angiopoietin-2, procalcitonin, fractalkine, pentraxin-3, and ST2.

126. A method of diagnosing and treating a bacterial infection in a human patient with a COVID-19 infection, comprising:
(a) providing a plasma sample from blood obtained from the human patient with the COVID-19 infection, wherein the plasma sample comprises microbial cell free nucleic acids (mcfNA)
(b) detecting the bacterial infection when an amount of bacterial mcfNA in the plasma sample exceeds a threshold amount of mcfNA; and
(c) administering a therapeutic drug to the human patient to treat the bacterial infection.

127. The method of claim 126, wherein the mcfNA is microbial cell-free DNA (mcfDNA).

128. The method of claim 126, wherein the therapeutic drug comprises one or more antibiotics.

129. The method of claim 128, wherein the one or more antibiotics comprises one or more antibiotics in Table 5.

130. The method of claim 126, wherein the COVID-19 infection is a first infection, and the bacterial infection is a secondary infection.

131. The method of claim 127, further comprising:
a. ligating adapters to microbial cfDNA (mcfDNA),
b. producing a sequencing library comprising mcfDNA attached to adapters,
c. detecting mcfDNA from at least two different microbes by aligning sequence reads generated from the massively parallel next generation sequencing to a database comprising bacterial reference sequence reads, wherein the at least two different microbes comprise bacteria from the group consisting of S. aureus, P. aeruginosa and K. pneumoniae, thereby detecting a bacterial infection;
d. administering one or more antibiotics to the human patient in order to treat the bacterial infection;

132. The method of claim 131, wherein the bacterial infection comprises a bacterial pneumonia caused by a respiratory pathogen listed in Table 2.
